(19) United States
(12) Patent Application Publication
(10) Pub. No.: US 2009/0220968 A1
(43) Pub. Date: Sep. 3, 2009

(54) Methods and Apparatus for Near Field Irradiation

(75) Inventors: David Issadore, Cambridge, MA (US); Thomas Hunt, Portland, OR (US); Kristi Adamson, Somerville, MA (US); Robert Westervelt, Lexington, MA (US); Rick Rogers, Needham, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 12/224,961

(22) PCT Filed: Mar. 9, 2007
(86) PCT No.: PCT/US2007/006103

§ 371 Date: Apr. 10, 2009

Related U.S. Application Data
(60) Provisional application No. 60/781,295, filed on Mar. 10, 2006.

(51) Int. Cl. G01N 33/53; H05B 6/80; H05B 6/72; C12Q 1/68; C12M 1/34

(52) U.S. Cl. 435/6; 219/679; 219/748; 219/750; 435/40.52; 435/7.1; 435/287.1

(57) ABSTRACT

Irradiation methods and apparatus configured to deliver power, via electromagnetic fields at a variety of frequencies and power levels, in a localized fashion to a target area. In one example, an electromagnetic field generator is disposed on a substrate and configured to deliver power via electromagnetic energy to a thin region proximate to (above) a surface of the substrate, wherein electromagnetic field intensity decreases significantly beyond the thin region. Such methods and apparatus are particularly useful in a wide variety of processes involving chemical and/or physical interactions in connection with a sample of interest located in the thin region. In different aspects, irradiator apparatus may be configured as disposable devices, and/or used in combination with one or more microfluidic or sensing components, for a variety of medical/laboratory/diagnostic methods and instrumentation implementations.

### PRIORITY

The present application claims priority to U.S. Provisional Application Ser. No. 60/781,295, filed Mar. 10, 2006, entitled “Methods and Apparatus for Near Field Irradiation,” which is hereby incorporated herein by reference.

### GOVERNMENT SPONSORED RESEARCH

Some of the research relating to the subject matter disclosed herein was sponsored by the United States National Science Foundation, award no. NSF-PHY-0117795, and the United States National Institute of Health, award no. NIH-1U54CA119349, and the United States government may have certain rights to some disclosed subject matter.

### BACKGROUND

A host of chemical and/or physical interactions involving a variety of sample types (including biological samples) may be enhanced, accelerated or otherwise affected by exposure to electric and/or magnetic fields having any of a number of different field strengths and frequencies/wavelengths throughout the electromagnetic spectrum.

For example, microwave enhanced chemistry is a well studied and accepted tool in a broad range of biological, medical, and chemistry fields. A great deal of investigation has gone into the optimization and study of reactions that use microwave radiation as an energy source in fields as far reaching as catalytic chemistry, solvent extraction, hydrolysis of proteins and peptides for amino acid analysis, and sample preparation in pathology. Microwave irradiation is a fundamentally different technique of inserting energy into chemical processes than conventional heating, and as such has added a great deal of unique results to many fields over its development.

An important application of microwave enhanced chemistry is in the field of biomedical histology, in which microwave driven fixation and staining is utilized to speed the analysis of thin slices of tissue gathered from surgical biopsy. Staining procedures have been developed using microwave irradiation which have reduced the processing time from 24 hours to a half of an hour. In such a procedure, thin slices of tissue may be fixated in protective paraffin, cut with a microtone a thickness of several microns, and stained for cancer cells in under an hour, making it possible to perform real time biopsies in explorative surgery.

The standard laboratory equipment for microwave irradiation is fundamentally the same as a conventional microwave oven used for cooking home food. A microwave oven works by passing microwave radiation, by convention at 2450 Megahertz (MHz), from a magnetron into a cooking chamber. The microwave radiation thusly generated in the cooking chamber provides energy to samples in the chamber. Although in many applications the samples of interest are very small volumes of fluid or very thin cuts of biological tissues (e.g., on the order of a few micrometers thick), large liter sized conventional microwave ovens remain the norm for all fields of microwave enhanced chemistry.

Again, in addition to microwave irradiation, there are several chemical and/or physical interactions involving a variety of sample types that may be enhanced, accelerated or otherwise affected by exposure to electric and/or magnetic fields at other frequencies in the electromagnetic spectrum. One area of the spectrum of particular interest includes radio frequency radiation. Whereas microwave (MW) radiation refers generally to electromagnetic radiation in the frequency range of approximately 300 MHz-300 gigahertz (GHz), radio frequency (RF) radiation refers generally to electromagnetic radiation in the frequency range of approximately 3 kilohertz (kHz)-300 Megahertz (MHz). Much research continues on possible biological effects of exposure to RF/MW radiation from a variety of sources, such as radios, cellular phones, the processing and cooking of foods, communications transmitters, radar transmitters, and the like.

### SUMMARY

With respect to the example of microwave irradiation discussed above, Applicants have recognized and appreciated that there are multiple problems associated with the use of conventional microwave ovens for laboratory purposes. For example, most chemical reactions require very exact temperature control; however lab microwaves have inherently poor power control. The magnetrons typically employed in conventional microwave ovens work only at a single power; therefore the power delivered to the sample can only be controlled by turning the power to the magnetron on and off. Water loading, in which a large cup of water is placed into the microwave to reduce the power delivered to the sample, is common practice in microwave-enhanced chemistry. Another inherent problem with microwave ovens is uneven heating. Uneven heating arises due to the complex standing wave patterns in which the microwaves fill the cooking chamber. The complex standing wave patterns are sensitive to the apparatus that holds the sample, and therefore expensive microwave transparent sample holders have become a prevalent laboratory product. Additionally, most work on microwave driven chemistry has been performed with irradiation at a frequency of 2450 MHz However, other frequencies within or beyond the microwave band, such as radio frequencies, may be of great interest. Finally, the size of samples of interest often is significantly smaller than the chamber of a conventional microwave oven.

In view of the foregoing, the present disclosure is directed generally to irradiation methods and apparatus that, in various embodiments, are configured to deliver power via electromagnetic fields at any of a variety of frequencies (e.g, radio frequency, microwave, other bands) and power levels in a localized fashion to a target area, such as the immediate vicinity of a sample of interest.

For example, in one embodiment, an apparatus according to the present disclosure comprises an electromagnetic field generator, or “irradiator,” disposed on a substrate. In various implementations, the substrate may be formed by a variety of rigid or flexible materials, and may have a variety of configurations including, but not limited to, planar, curved, bent, circular, conical, tubular, well-shaped, and others. In exemplary aspects, the apparatus may be configured to deliver on the order of milliwatts of power (e.g., 0 to approximately 100 mW) via electromagnetic energy to a thin region (e.g., up to on the order of approximately 100 micrometers or greater) proximate to (above) a surface of the substrate. However, it should be appreciated that the apparatus is not limited in these respects, as different irradiation powers and regions are possible according to various embodiments. Generally, the apparatus produces a thin layer of intense electromagnetic field intensity that falls off exponentially in distance away from the substrate.

In various embodiments, different irradiator geometries are configured to excite electric and/or magnetic near-field modes. The ability to independently excite electric and magnetic modes may be used for selective irradiation of various sample types. For example, an irradiator apparatus configured to generate electric fields in a localized target area (thin region) proximate to the apparatus may be used to provide dielectric heating to a sample in the target area. Peak absorption frequencies of different samples may depend at least in part on the nature of the irradiated sample (e.g., organic molecules and tissues that confine water, aqueous protein solutions, etc.). An irradiator apparatus configured to generate magnetic fields in a localized target area may be used to selectively heat materials impregnated with magnetic particles (e.g., magnetic nanoparticles).

Generally, irradiator apparatus and methods according to the present disclosure provide local and rapid irradiation of samples disposed in the irradiated target area. Such methods and apparatus are particularly useful in a wide variety of processes involving chemical and/or physical interactions in connection with the sample of interest; in particular, samples with small volumes may be irradiated evenly and efficiently, over a range of frequencies and power levels. Moreover, in other aspects, irradiator apparatus according to the present disclosure may be made inexpensively, and in some cases may be implemented as disposable devices. In yet other embodiments, irradiator apparatus of the present disclosure may be used in combination with one or more microfluidic components and/or sensors, for example, in a variety of medical diagnostic instrumentation implementations.

In sum, one embodiment is directed to an apparatus, comprising a substrate, and at least one electromagnetic field generator disposed on the substrate, wherein the at least one electromagnetic field generator, when energized, is configured to deliver power only to a localized area comprising a thin region proximate to the substrate.

Another embodiment is directed to an electromagnetic irradiation method, comprising an act of delivering power only to a localized area comprising a thin region proximate to a substrate.

Another embodiment is directed to a method for accelerating or enhancing a chemical process. The method comprises: obtaining a biological sample; contacting the biological sample with a reagent or reagents required for performing the chemical process; and subjecting the biological sample to an electromagnetic field localized to the immediate vicinity of the biological sample, the electromagnetic field providing a level of power and the biological sample being subjected for a duration of time sufficient to achieve such acceleration or enhancement of the chemical process.

Another embodiment is directed to a method of accelerating or enhancing a binding assay. The method comprises: obtaining a test sample; contacting the test sample with a target compound; and, subjecting a mixture containing the test sample and the target compound to an electromagnetic field localized to the immediate vicinity of the mixture, the electromagnetic field providing a level of power and the mixture being subjected for a duration of time sufficient to achieve such acceleration or enhancement of the binding assay.

Another embodiment is directed to the use of an apparatus for accelerating or enhancing a process of intermolecular interaction in a sample, wherein the apparatus comprises a substrate; and an electromagnetic field generator deposited on the substrate for irradiating a localized region within an immediate vicinity of the sample.

It should be appreciated that all combinations of the foregoing concepts and additional concepts discussed in greater detail below are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

FIG. 1(a) illustrates various concepts in connection with an irradiator apparatus according to one embodiment of the present disclosure.

FIGS. 1(b) and 1(c) are graphs of computed electric field contours for two exemplary irradiator apparatus according to embodiments of the present disclosure.

FIG. 2(a) illustrates a top view of an irradiator apparatus according to another embodiment of the present disclosure having a coiled transmission line configuration.

FIG. 2(b) is a cross-sectional side view of a portion of the apparatus shown in FIG. 2(a).

FIG. 3 illustrates a top view of an irradiator apparatus according to another embodiment of the present disclosure.

FIG. 4 illustrates a top view of an irradiator apparatus configured to generate localized magnetic fields according to another embodiment of the present disclosure.

FIG. 5 illustrates a method of irradiating a thin tissue according to one embodiment of the present disclosure.

FIG. 6 illustrates an exemplary cross-sectional schematic of a configuration involving an irradiator device and sample slide used in the method of FIG. 5.

FIG. 7 is a schematic showing exemplary experimental steps that may be enhanced by the present disclosure.

### DETAILED DESCRIPTION

To demonstrate some fundamental concepts underlying an irradiation apparatus according to one embodiment of the present disclosure, the behavior of electric and magnetic fields is considered for a periodic series of conductors (e.g., electrodes or wires). For purposes of illustration, the electrical case is considered in detail, but it should be appreciated that the mathematical analysis outlined below is analogous for magnetic fields.

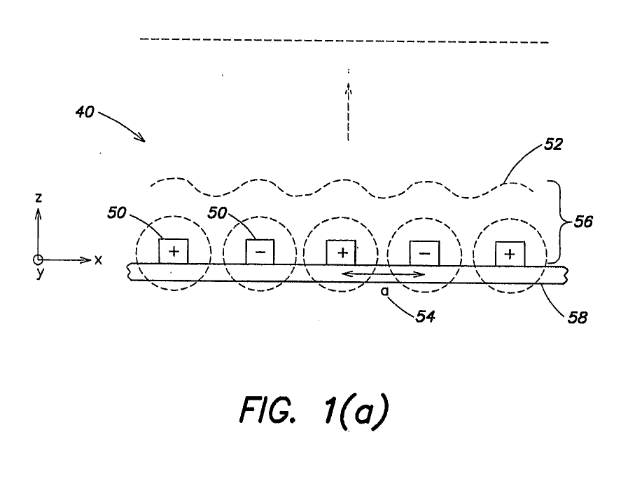

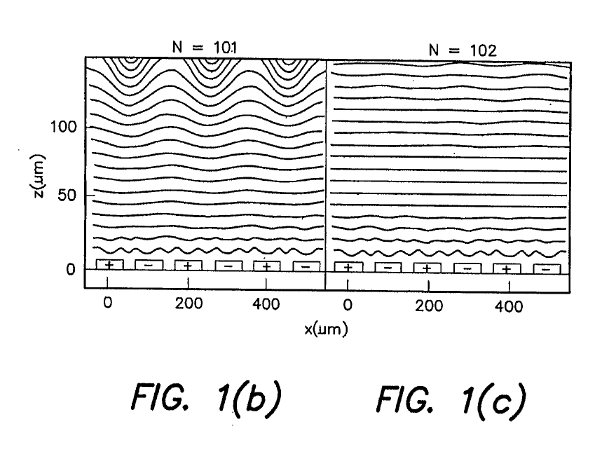

First, an idealized case for an irradiator apparatus 40 according to one embodiment of the present disclosure is considered in FIG. 1(a). The irradiator apparatus 40 shown in FIG. 1(a) comprises conductors 50 disposed on a substrate 58 and arranged to form a parallel array of parallel equally-spaced conductors in an x-y plane defined by the substrate, wherein adjacent conductors have an opposite polarity (e.g., an equal and opposite voltage is applied to adjacent conductors). To facilitate preliminary analysis, for the moment the conductors are considered to be infinitely long in they-direction and repeated infinitely in parallel along the x-direction. As a result of the voltage applied to the conductors, an electric field is generated in the vicinity of the conductors above the substrate. If the generated field is examined at a large distance in the z-direction normal to the x-y plane, it is found that the field is zero. In particular, the conductors with opposite potential cancel each other such that there are no electric field lines at large distances. As one moves close to the array of conductors, there is a non-zero spatially varying field 52 in a thin region 56 proximate to the substrate 58 that gets stronger as the distance above the array decreases.

To calculate the field 52 at distances close to the array, i.e., in the thin region 56, due to the periodicity of the array the field 52 may be expressed in terms of a potential constituted by a sum of periodic functions in a Fourier series, given by:

$$φ ( x , z ) = {F}_{n} ( z ) cos \frac{2 π nx}{a} , \qquad ( 1 )$$

where ø represents the potential as a function of x and z, x denotes position along the array parallel to the plane of the array, z denotes the distance from and normal to the plane of the array, α is the spacing 54 between adjacent conductors, and n designates the mode of the Fourier series. Noting that in the regions above the array there is no net charge, the potential must satisfy Laplace's equation:

$$\begin{matrix}\frac{{\partial }^{2} φ}{\partial {x}^{2}} + \frac{{\partial }^{2} φ}{\partial {z}^{2}} = 0 ; & ( 2 ) \\ - \frac{4 {π}^{2} {n}^{2}}{{a}^{2}} {F}_{n} ( z ) cos \frac{2 π nx}{a} + \frac{{d}^{2} {F}_{n}}{d {z}^{2}} cos \frac{2 π nx}{a} = 0. & ( 3 )\end{matrix}$$

With the constraint of Laplace's equation, the harmonics of the Fourier components of the field drop off as an exponential with a characteristic distance:

$$\begin{matrix}{F}_{n} = {A}_{n} {e}^{- z / {z}_{o}} ; & ( 4 ) \\ {z}_{o} = \frac{a}{2 π n} . & ( 5 )\end{matrix}$$

Accordingly, with a periodic array of conductors at alternating opposite potentials, it is observed that the electrostatic potential drops off at a characteristic distance based on the spacing 54 (also referred to as pitch or period) of the conductors. Hence, the extent of the thin region 56, normal to the substrate, is determined at least in part by the conductor spacing 54. In some exemplary embodiments, the value a may be particularly selected such that the characteristic distance for the thin region may fall in a range of from approximately 1 micrometer (beyond which the field falls off sharply), to hundreds of micrometers (beyond which the field falls off sharply).

Thus, according to various embodiments, irradiator apparatus contemplated herein operate utilizing the foregoing principals to create oscillating electric or magnetic fields whose intensity drops off very sharply beyond a characteristic distance that delimits a thin region proximate to a substrate on which the conductors of the apparatus are disposed. Hence, a given apparatus irradiates only a thin layer proximate to the substrate, without wasting energy by radiating out to the universe.

In one embodiment, an irradiator apparatus according to the present disclosure based on-the concepts illustrated in FIG. 1 (a) comprises a number N of conductors 50 having a finite length in the y-direction and disposed on a substrate 58 in a parallel equally-spaced manner along the x-direction. In one exemplary implementation, N may be on the order of 100, the substrate may be glass, and the overall dimensions of the irradiator apparatus in the x-y plane may be on the order of 1 cm , wherein each conductor has a width along the x-direction of approximately 70 mm, a height normal to the substrate in the z-direction of approximately 7 mm, and a spacing 54 (“a” in the equations above) of approximately 200 mm. It should be appreciated that the foregoing examplary parameters are provided primarily for purposes of illustration, and that irradiator apparatus according to other embodiment of the present disclosure are not limited to the various parameters associated with this particular example.

For the exemplary parameters above, fall-off of the electric field 52 generated by the irradiator apparatus in the z-direction is shown in a finite-element simulation in FIGS. 1 (b) and 1 (c). In particular, FIG. 1 (b) shows the computed electric field contours for an odd number N of electrodes (in the illustrated example, N=101), whereas FIG. 1(c) shows similar contours for an even number of electrodes (e.g., N=102). The array of conductors is centered at x=0, and z is the distance from and normal to the plane of the substrate. Subsequent contours are the ratio Eₙ₊₁/Eₙ=0.8, where n=1 to 20 (n designates modes of the Fourier series in the above equations). From FIG. 1(c), it may be appreciated that for an even number of electrodes (i.e., for every positive potential there is an opposite negative potential), the field goes sharply to zero beyond a localized area comprising a thin region proximate to (e.g., above) the substrate (in the figures associated with these particular examples, on the order of 50 micrometers).

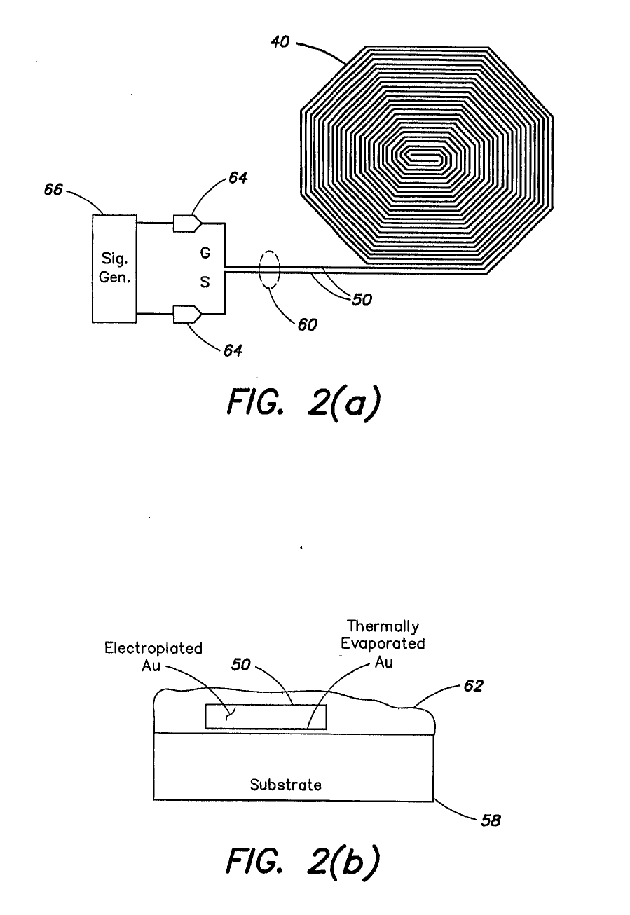

In another embodiment, the electric mode variant of an irradiator apparatus 40 comprises conductors forming a transmission line 60 (two parallel metal lines) that coils about in the shape of an octagon, as illustrated in FIG. 2(a). In an exemplary implementation of this embodiment, the coiled transmission-line irradiator apparatus 40 may be fabricated on a substrate 58 formed by a standard 1″ by 3″ glass slide, although as discussed above it should be appreciated that a variety of other substrates generally may be suitable. A cross-sectional diagram of such a device is shown in FIG. 2(b). In one aspect, the octagon-shaped coil is configured such that the irradiation region is approximately 8 millimeters×8 millimeters parallel to the plane of the substrate.

As discussed above in connection with equations (1)-(5), various spacings a between the metal lines may be chosen to achieve a desired extent of a thin region proximate to the substrate in which power is delivered to a sample. In various implementations, the spacing or pitch of the conductors may be selected such that this region in which power is delivered ranges from approximately one micrometer to hundreds of micrometers in a direction normal to the plane of the transmission line coil. In one example, metal lines having a width of approximately 100 micrometers, with a spacing between metal lines of approximately 100 micrometers, form an irradiator apparatus similar to that shown in FIG. 2(a).

The metal lines may be defined by liftoff of a metal layer (10 nanometers titanium (Ti), 40 nanometers gold (Au)) following photolithographic patterning. A thick (5 μm) layer of gold subsequently may be electroplated onto the metal lines with a gold plating solution, stirred at 65° C., with a deposition rate of approximately 5 micrometers/hour. By such electroplating, the lines are thickened so as to mitigate ohmic heating. Additionally, according to another aspect of this embodiment, a thin conformal layer 62 (approximately 1 micrometer thick) of Teflon may be spun onto the apparatus to reduce adhesion between the sample to be irradiated (or material containing the sample) and the apparatus. More generally, any appropriate suface coating may be employed to reduce or prevent nonspecific binding or adherence of samples or solutions containing samples to the apparatus itself. Other examples of such coatings include, but are not limited to, a thin filmlayer/coating on the order of micrometers comprising Mylar film, epoxy, nonconductive silicone rubber, or silicone grease.

In other aspects of this exemplary implementation, the irradiator apparatus shown in FIG. 2(a) may include electrical contacts in the form of two 1 millimeter by 1 millimeter contact pads 64, for example. The apparatus may be driven by a signal generator 66 that can provide various signal power levels (e.g., on the order of up to 20 dBm). In one embodiment, the signal generator 66 may be implemented as a printed circuit board circuit that may be integrated with or coupled to the substrate. A flip-chip pressure connector may be used to couple the signal generator to the irradiator so as to remove the complication of wires that may become a power delivery problem at high frequencies.

In yet other implementations, a printed circuit (PC) board may be employed as a substrate on which the conductors of an irradiator apparatus are formed (e.g. coiled transmission line configuration), and the conductors may be formed of materials other than titanitum/gold (e.g., copper, lead-coated copper, etc.). As indicated above, irradiator apparatus formed on a PC board substrate optionally may be coated with a layer of epoxy or other coating to reduce/prevent adhesion between the apparatus and the sample/solution containing sample.

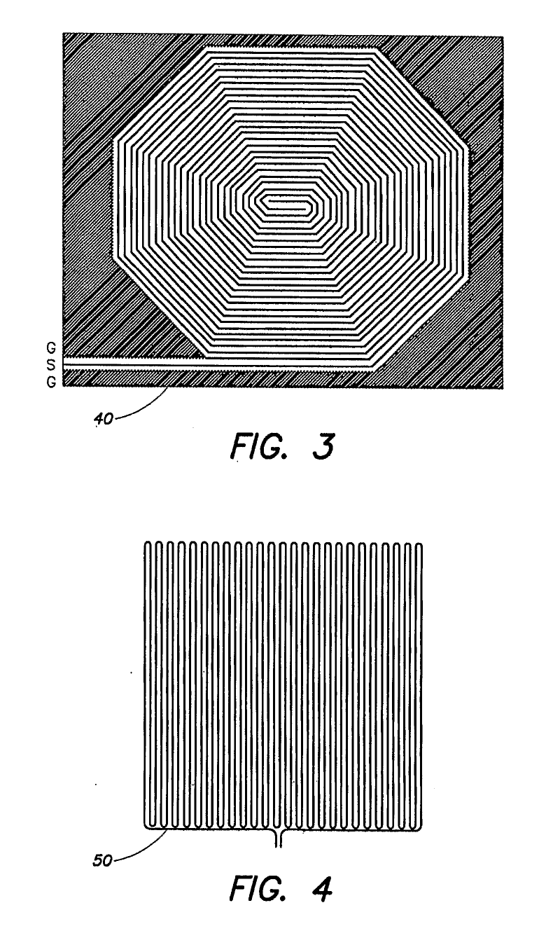

With respect to electrical signals applied to irradiator apparatus according to the present disclosure; for signals that have a wavelength much larger than the size of the field generating components of an irradiator apparatus (i.e., signals from DC to approximately 500 MHz) a quasi-static approximation may be made, such that the DC analysis may be applied to the behavior of the apparatus. For higher frequency signals approaching the gigahertz (GHz) range (e.g., microwave radiation), the wavelengths of the electromagnetic radiation may approach the same size scale as the dimensions of conductors used for the irradiator apparatus, and impedance matching between the signal generator and the irradiator apparatus may become important. Accordingly, to improve impedance matching into the GHz range, FIG. 3 illustrates the coiled design of FIG. 2(a) implemented with ground-source ground terminals.

According to another embodiment, a magnetic mode variant of an irradiator apparatus may comprise a length of wire that coils about itself in a serpentine pattern, as is shown in FIG. 4. Magnetic fields do not couple well to electric dipoles, and as such a magnetic mode irradiator generally has poor heating efficiency for non-magnetic materials. However, such an irradiator can couple very strongly to magnetic particles (e.g., mangetic nano-particles), and as such has excellent selectivity for objects impregnated with magnetic nano-particles. As above, with respect to an exemplary fabrication process, the metal lines may be defined by liftoff of a metal layer (10 nm Ti, 40 nm Au) following photolithographic patterning. A thick (5 μm) layer of gold may be electroplated onto the metal lines with a gold plating solution, stirred at 65° C., with a deposition rate of ˜5 μm/hr. A thin conformal layer (˜1 μm) of Teflon also may be spun onto the apparatus to reduce adhesion of biomaterial to the apparatus.

According to yet another embodiment, an additional modality of the apparatus disclosed herein includes applying DC offset to the excitation signal applied from the signal generator to the irradiation apparatus. A DC offset voltage may be applied in linear superposition to the AC field, and can be adjusted to a specific proteins isoelectric point, tuned to drive antibodies in solution onto tissues or target binding sites. Similar to isoelectric focusing based on exact pH characteristics, proteins can be driven out of solution to their targets based on the application of an appropriate DC offset.

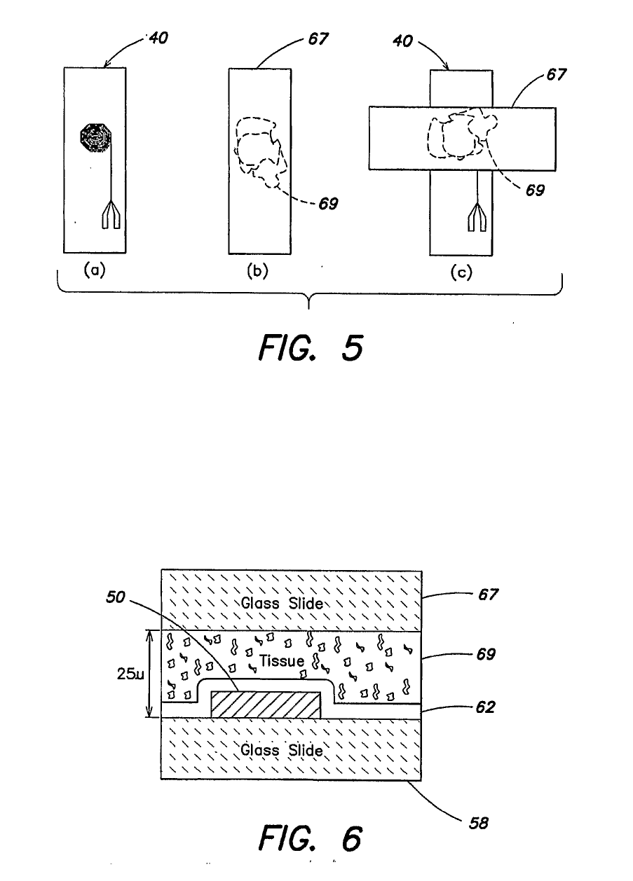

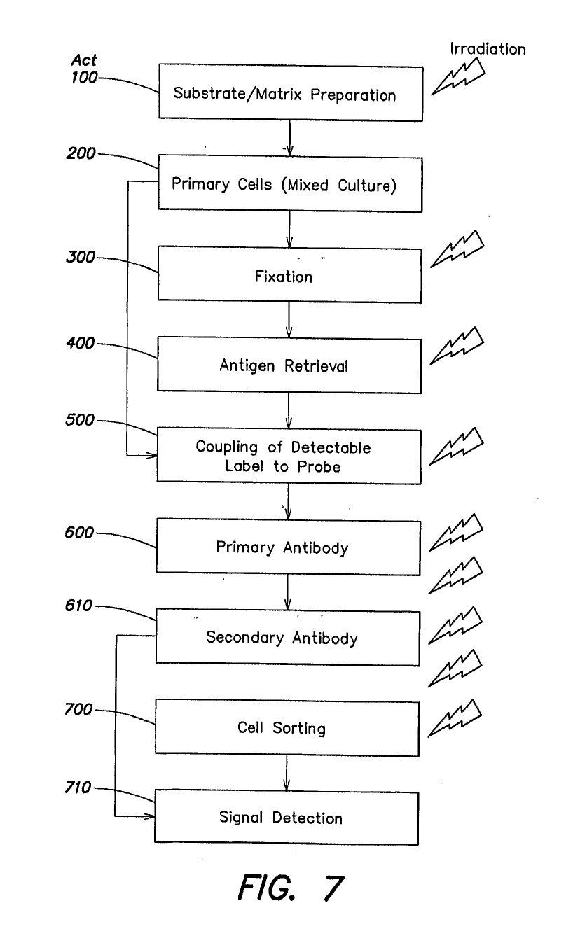

In one exemplary application, irradiators according to the present disclosure may be used in the enhanced fixation and staining of tissues with bio-markers. This is illustrated in FIG. 7 (Act 300). As discussed above, microwave enhanced fixation and staining is a common procedure in histology, to date involving large conventional microwave ovens which may be replaced by irradiators pursuant to the concepts disclosed herein, operating at a variety of possible frequency ranges (e.g., microwave, radio frequency, other bands). The illustrations of FIGS. 5 and 6 outline how such irradiators may be employed to deliver power via electromagnetic radiation to a tissue. In particular, FIG. 5(a) shows an irradiator apparatus 40 implemented on a glass slide substrate, FIG. 5(b) shows a tissue sample 69 disposed on a second glass slide substrate 67, and FIG. 5(c) shows the tissue sample/glass slide overlaying the irradiator apparatus 40 in a criss-cross manner. FIG. 6 illustrates a portion of a cross section of this arragnement, in which one exemplary conductor 50 of the irradiator apparatus 40 is placed in close proximity to the tissue 69, such that the tissue is located in the thin region to which the irradiator apparatus delivers power.

Accordingly, methods and apparatus according to the present disclosure are useful for a wide range of biological and medical procedures. A number of such applications are contemplated, including, inter alia, methods directed to biochemical, histochemical, histopathological, biomedical, and analytical uses.

The methods and apparatus disclosed herein are useful for improving one or more aspects of a variety of routine analytical and histological procedures employed in research and clinical laboratories, as well as in medical/clinical practice. In one aspect, the various concepts disclosed herein provide methods for accelerating or enhancing chemical processes. As used herein, “chemical processes” shall encompass histological processes, histochemical processes, cytochemical processes, immunochemical processes, immunohistochemical processes, immunocytochemical processes, colometric processes, chemical processes involving nanoparticles, electrochemical processes, etc.

In typical embodiments, the methods involve obtaining a biological sample to be analyzed or histologically processed, performing an appropriate histochemical process or processes using a suitable reagent or reagents, and during one or more steps of such procedures, allowing the biological sample to be exposed to an electromagnetic field defined herein. The degree (intensity/level and duration) to which the biological sample is subjected to the electromagnetic field will depend on a number of factors, such as the type of the biological sample, thickness of the sample (e.g., tissue sections), the nature of the histological process, intrinsic sensitivity of the assay or procedures being performed, and so on. In general, histochemical processes of biological samples include multiple steps, such as fixation, staining, incubations, washing, etc. Thus, the present invention may be applied to one or more of these steps to improve general outcome of chemical, and/or related analytical procedures.

As used herein, the terms “accelerating” “accelerate” and “accelerating” shall mean that the amount of time required to obtain reasonably reliable outcome that is equivalent in quality as obtained by conventional methods is shortened. For example, a staining process that typically requires by conventional methods several hours to overnight may be reduced to in an order of seconds to minutes by the methods disclosed herein. Similarly, each of multiple incubation and intervening washing periods associated with a typical chemical procedure may be shortened significantly using the methods of the invention.

The terms “enhancing” “enhance” and “enhancement” refer to improvement in the overall quality of a product, process, and/or data, as compared to conventional methods that are available. For example, data acquired according to one or more embodiments of the present invention may be enhanced by a heightened signal-to-noise ratio. That is, the methods described herein may increase a specific signal and/or reduce background (or noise) so that the resulting products, processes and/or data are of better quality. Using the methods provided herein, the invention also allows generating comparable results using significantly less volume of reagents required for performing one or more steps of these processes. As a result, the invention may realize significant cost reduction, particularly in situations where a large number of samples are processed, or in cases where reagents are limited in quantity or costly. Depending on the particular sample, the nature of the technique, and also depending on the type of substrate being used, a typical reaction may require a reagent volume of in the order of microlitters—such as 1, 2, 5, 10, 25, 50, 100 microliters. In certain embodiments of the invention, the histochemical processes described herein shall embrace immunohistochemical processes.

Immunohistochemistry involves the localization of antigens in a cell or tissue section by the use of labeled antibodies as specific reagents through antigen-antibody interactions that are visualized by a marker such as fluorescent dye, enzyme, radioactive element, colored dye, marker, stain or colloidal gold. Therefore, immunohistochemistry has become a crucial technique and widely used in many medical research laboratories as well as clinical diagnostics. The technique offers a wide range of variations and modified protocols, which the art is familiar with. The selection of a suitable method should be based on parameters such as the type of specimen under investigation and the degree of sensitivity required. A skilled partisan will be able to determine a suitable application in incorporating the methods and uses taught in the invention as disclosed herein.

In some embodiments, the methods of the invention are used for histochemical processes involving a cross-linking process. Cross-links are covalent bonds linking one polymer chain to another. In biology, cross-linking has applications in forming polyacrylamide or agarose gels for gel electrophoresis in studies of proteins and/or nucleic acids, as well as other matrices including those used as a substrate for cell culture and tissue engineering. The term also encompasses cross-linking compounds that are used to selectively couple a chemical constituent of a moleule. For example, a variety of crosslinker are used to study subunit conformation of proteins. This is deduced since crosslinkers only bind surface amino residues in relatively close proximity in the native state. Examples of crosslinkers are dimethyl suberimidate and glutaraldehyde. Both induce nucleophilic attack of the amino group of lysine and their subsequent covalent bonding via the crosslinker. However, the methods described herein may be useful for any other chemical crosslinkers.

In yet other cases, however, cross-linking may involve more general “fixing” such as fixation of a cell or tissue for primarily preservation purposes. In the fields of histology, pathology, and cell biology, fixation is a chemical process by which biological tissues are preserved from decay. Fixation terminates any ongoing biochemical reactions, and may also increase the mechanical strength or stability of the treated tissues. Thus, the main purpose of fixation is to preserve a sample of biological material, such as tissue or cells, to permit stable storage and analysis. To achieve this goal, several conditions must usually be met. First, a fixative usually acts to disable intrinsic biomolecules—particularly proteolytic enzymes—which would otherwise digest or otherwise damage the sample. Second, a fixative will typically protect a sample from extrinsic damage. Many fixatives are toxic to most common microorganisms (bacteria in particular) which might exist in a tissue sample or which might otherwise colonize the fixed tissue. In addition, many fixatives will chemically alter the fixed material to make it less palatable (either indigestible or toxic) to opportunistic microorganisms. Finally, fixatives often alter the cells or tissues on a molecular level to increase their mechanical strength or stability. This increased strength and rigidity can help preserve the morphology of the sample as it is processed for further analysis. Fixation is usually the first stage in a multistep process to prepare a sample of biological material for microscopy or other analysis. Therefore, the choice of fixative and fixation protocol will depend heavily on the additional processing steps and final analyses that are planned. For example, immunohistochemistry utilises antibodies which bind to a specific protein target. The use of the present invention is not limited to a particular fixative or histochemical procedure, and thus may be adapted for use in conjunction with any of the methods described herein and the like.

Crosslinking fixatives act by creating covalent chemical bonds between proteins in tissue. This anchors soluble proteins to the cytoskeleton, and lends additional rigidity to the tissue. Accordingly, the present invention contemplates improving aspects of such fixation procedures (by accelerating or enhancing the process) that are commonly employed. In some embodiments, the invention is used for histochemical process involving the crosslinking fixative, formaldehyde (often sold as a saturated aqueous solution under the name formalin). Formaldehyde is thought to interact primarily with the residues of the basic amino acid lysine. In some embodiments, the invention is used with glutaraldehyde. While it is believed to operate by a similar mechanism to formaldehyde, as a somewhat larger molecule, glutaraldehyde may not penetrate thicker tissue specimens as effectively as formaldehyde. On the other hand, glutaraldehyde may offer a more rigid or tightly linked fixed product—its greater length and two aldehyde groups allow it to ‘bridge’ and link more distant pairs of protein molecules. In yet other cases, fixation protocols call for a combination of formaldehyde and glutaraldehyde, so that their respective strengths complement one another. Examples of common fixative solutions used for immunohistochemistry include the followings: (a) 4% paraformaldehyde in 0.1 M phosphate buffer; (b) 2% paraformaldehyde with 0.2% picric acid in 0.1 M phosphate buffer; (c) PLP fixative: 4% paraformaldehyde, 0.2% periodate and 1.2% lysine in 0.1 M phosphate buffer; and (d) 4% paraformaldehyde with 0.05% glutaraldehyde (electron microscopy immunohistochemistry). However, it is understood that a skilled partisan may make modifications to optimize conditions to suit a particular use.

Yet in other embodiments, oxidizing agents are used. The oxidising fixatives can react with various side chains of proteins and other biomolecules, allowing the formation of crosslinks which stabilize tissue structure. For example, osmium tetroxide is often used as a secondary fixative when samples are prepared for electron microscopy. Potassium dichromate, chromic acid, and potassium permanganate all find use in certain specific histological preparations.

The invention may be used for fixation procedure involving fixatives which are characterized as precipitating fixatives. Precipitating (or denaturing) fixatives act by essentially reducing the solubility of protein molecules and often by disrupting the hydrophobic interactions which give many proteins their tertiary structure. The precipitation and aggregation of proteins is a very different process from the crosslinking which occurs with the aldehyde fixatives. The most common precipitating fixatives include ethanol and methanol. Acetone is also used.

Acetic acid is a denaturant that is sometimes used in combination with the other precipitating fixatives. The alcohols, by themselves, are known to cause shrinkage of tissue during fixation while acetic acid alone is associated with tissue swelling; combining the two may result in better preservation of tissue morphology. In certain circumstances, the invention may be also used in a fixation process using fixative agents that contain picric acid and mercuric chloride. In any of the above situations, the methods desclosed herein may accelerate and/or enhance the process of fixation.

Similarly, the invention finds applications in improving chemical or histochemical processes involving staining. Stains and dyes are frequently used in biology and medicine to highlight structures in biological tissues for viewing, often with the aid of different microscopes. Stains may be used to define and examine bulk tissues (highlighting, for example, muscle fibers or connective tissue), cell populations (classifying different blood cells, for instance), or organelles within individual cells. Thus, staining is a biochemical technique of adding a class-specific (DNA, proteins, lipids, carbohydrates) dye to a substrate to qualify or quantify the presence of a specific compound. For example, biological staining can be used to mark cells in flow cytometry, and to flag proteins or nucleic acids in gel electrophoresis. Thus, the invention in some embodiments embraces methods for accelerating and/or enhancing these procedures.

There are a number of staining processes that may benefit from the invention disclosed herein. Not intending to be limiting, these include: Acid Fast Bacilli Staining, Alcian Blue Staining, Alcian Blue/PAS Staining, Alizarin Red Staining, Alkaline Phosphatase Staining, Azure A Staining, Bielschowsky Staining, Congo Red Staining, Diff-Quik Staining, Diff-Quik II Stain for Helicobacter pylori, Fite Faraco Staining, Giemsa Staining, Golgi Staining, Golgi-Cox Staining, Gomori's Trichrome Staining, Gordon Sweet's Staining, Gram Staining, Grocott Methenamine Staining, Haematoxylin and Eosin Staining, Hyaluronidase Alcian Blue Staining, Luna Staining, Luxol Fast Blue Staining, Masson Fontana Staining, Masson Trichrome Staining, Methenamine Sliver Staining, Microglia Staining, Miller's Elastic Staining, Nissl Staining, Oil Red O Staining, PAS Staining, PAS Diastase Staining, Perls Prussian Blue Staining, Pouchet Staining, Prussian Blue Staining, Renal Alcian Blue/PAS Staining, Renal Masson Trichrome Staining, Renal PAS Methenamine Staining, Rhodanine Staining, Safranin O Staining, Sirius Red for Collagen Staining, Southgate's Mucicannine Staining, Toluidine Blue Staining, van Gieson Staining, von Kossa Staining, VVG Staining, X-Gal Staining and Ziehl Neelsen Staining. Some are further discussed below.

The amount of time required for completing a staining process greatly varies, but in any case, the overall process may be accelerated when the present invention is applied. At its simplest, the actual staining process may involve immersing the sample (before or after fixation and mounting) in dye solution, followed by rinsing and observation. Many dyes, however, require the use of a mordant: a chemical compound which reacts with the stain to form an insoluble, coloured precipitate. When excess dye solution is washed away, the mordanted stain remains. Gram staining is used to determine gram status to classify bacteria broadly. It is based on the composition of their cell wall. Gram staining uses crystal violet to stain cell walls, iodine as a mordant, and a fuchsin or safranin counterstain to mark all bacteria. Gram status is important in medicine; the presence or absence of a cell wall will change the bacterium's susceptibility to some antibiotics. Gram-positive bacteria stain dark blue or violet. Their cell wall is typically rich with peptidoglycan and lacks the secondary membrane and lipopolysaccharide layer found in Gram-negative bacteria. These differential characteristics, therefore, may aid a histopathological analysis and subsequent diangnosis of a disease or disorder. Accordingly, the present invention may accelerate such process. Haematoxylin and eosin staining protocol is used frequently in histology to examine thin sections of tissue, and thus a useful tool in pathology. Haematoxylin stains cell nuclei blue, while eosin stains cytoplasm and connective tissue pink or red. Thus, the invention includes methods for speeding up a process of Haematoxylin staining of a patient specimen, for example, during a surgery. In addition, Eosin is strongly absorbed by red blood cells, colouring them bright red. Such property may be used in analyzing blood samples. Applying this to the present inevntion, it is possible to greatly improve such analytical procedures.

Papanicolaou staining, or Pap staining, is a frequently used method for examining cell samples from various bodily secretions. It is frequently used to stain the Pap smear specimens. In general, it uses a combination of haematoxylin, Orange G, eosin Y, Light Green SF yellowish, and sometimes Bismarck Brown Y. In some embodiments, therefore, the invention contemplates accelerating and/or enhancing the process of Pas smear tests. For example, the invention may realize an in-visit Pap smear test, where a patient may obtain a result of a test during a single visit to her physician's office, as opposed to receiving a result on a later date.

Similarly, Periodic acid-Schiff staining (PAS staining) is used for demonstrating carbohydrates (including glycogen, glycoprotein, proteoglycans). It is used to distinguish different types of glycogen storage diseases. Therefore, some embodiments of the invention relate to improving the process of diagnosing and/or monitoring such diseases, based on more rapid PAS staining.

In some embodiments, the invention is used for applications involving staining protocols for Masson's trichrome, which is a three-colour staining protocol well-suited to distinguish cells from surrounding connective tissue. Most recipes will produce red keratin and muscle fibers, blue or green staining of collagen and bone, light red or pink staining of cytoplasm, and black cell nuclei.

Yet in other embodiments, the methods are provided to enhance staining process of the Romanowsky stains, which are all based on a combination of eosinate (chemically reduced eosin) and methylene blue (sometimes with its oxidation products azure A and azure B). Common variants include Wright's stain, Jenner's stain, Leishman stain and Giemsa stain. All can be used to examine blood or bone marrow samples. They are generally preferred over H&E for inspection of blood cells because different types of leukocytes (white blood cells) can be readily distinguished. All are also suited to examination of blood to detect blood-borne parasites like malaria.

In certain cases, the methods provided herein may be applied to Silver staining, in which silver is used to stain histologic sections. This kind of staining is important especially to show proteins (for example type III collagen) and DNA. It is used to show both substances inside and outside cells. For instance, some cells are argentaffin. These reduce silver solution to metallic silver after forrnalin fixation. This method is based on a reaction between silver nitrate and potassium dichromate, thus precipitating silver chromate in some cells. Other cells are argyrophilic. These reduce silver solution to metallic silver after being exposed to the stain that contains a reductant, for example hydroquinone or formalin.

Still in other cases, the invention provides methods for improving Sudan staining. Sudan staining takes advantage of Sudan dyes to stain sudanophilic substances, usually lipids. Sudan III, Sudan IV, Oil Red O, and Sudan Black B are often used. Sudan staining is often used to determine the level of fecal fat to diagnose steatorrhea. Thus, the methods according to the present invention can significantly speed up the process.

In certain embodiments, the invention is useful for improving in vivo staining. In vivo staining is the process of dyeing living cells or tissues. By causing certain cells or structures to take on contrasting color(s), their. morphology or position within a cell or tissue can be readily seen and studied. The usual purpose is to reveal cytological details that might otherwise not be apparent; however, staining can also reveal where certain chemicals or specific chemical reactions are taking place within cells or tissues. As would be clear to those skilled in the art, such methods can offer valuable advantage for a number of clinical and analytical applications.

Often these stains are called vital stains. They are introduced to the organism while the cells are still living. However, these stains are eventually toxic to the organism, some more so than others. To achieve desired effects, the stains are used in very dilute solutions ranging from 1:5,000 to 1:500,000. Note that many stains may be used in living cells, such as primary cells grown in culture.

There are many effective biological stains available in the art. Different stains react or concentrate in different parts of a cell or tissue, and these properties are used to advantage to reveal specific parts or areas. Generally, these dyes may be used with fixed cells and tissues, and some are particularly suitable for use with living organisms (“vital dyes”). Non-limiting examples of biological stains that are commonly used include: Bismarck brown, Carmine, Coomassie blue, Crystal violet, DAPI, Eosin, Ethidium bromide, Fuchsin, Haematoxylin, Hoechst stains, Iodine, Malachite green, Methyl green, Methylene blue, Neutral red, Nile blue, Nile red, Osmium tetroxide, Rhodamine, Safranin.

Similar to light microscopy, stains can be used to selectively highlight cellular structures in transmission electron microscopy, and thus the present invention also includes methods of acclerating and/or enhancing one or more steps of preparing biological samples for EM analysis. Electron-dense compounds of heavy metals are typically used. For example, phosphotungstic acid is a common negative stain for viruses, nerves, polysaccharides, and other biological tissue materials. Other chemicals used in electron microscopy staining include ammonium molybdate, cadmium iodide, carbohydrazide, ferric chloride, hexarnine, indium trichloride, lanthanum nitrate, lead acetate, lead citrate, lead(II) nitrate, osmium tetroxide, periodic acid, phosphomolybdic acid, potassium ferricyanide, potassium ferrocyanide, Ruthenium Red, silver nitrate, sodium chloroaurate, thallium nitrate, thiosemicarbazide, uranyl acetate, uranyl nitrate, and vanadyl sulfate.

Throughout the application disclosed herein, where so desired, immunodetection may be carried out by any number of available protocols of choice, which will benefit when used in conjunction with the methods provided herein. Target detection used in any of the methods of the present invention as described herein, including chemical assays, histochemical processes, immuno-affinity assays, binding assays, screenings, and the like, generally employs detectable label or labels, which are either colorimetric or fluorometric in nature, or combination thereof. In certain circumstances, these detectable labels are nano-particles. For example, fluorescent nano-particles conjugated to a primary or secondary antibody, for instance, are particularly advantagous reagents since they do not fade after exposure to fluorescent light, whereas many chemical dyes commonly do. An exemplary immunohistochemical procesure is outlined in FIG. 7.

Common fluorophores include but are not limited to: 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodarnine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange+DNA; Acridine Orange+RNA; Acridine Orange, both DNA & RNA; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC=Ratio Dye, Zn2+; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET Bimane; Bisbenzamide; Bisbenzimide (Hoechst); bis-BTC=Ratio Dye, Zn2+; Blancophor FFG; Blancophor SV; BOBO™-1; BOBO™-3; Bodipy 492/515; Bodipy. 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC-Ratio Dye Ca2+; BTC-SN-atio Dye, Zn2+; Calcein; Calcein Blue; Calcium Crimson™; Calcium Green; Calcium Green-1 Ca2+Dye; Calcium Green-2 Ca2+; Calcium Green-5N Ca2+; Calcium Green-C18 Ca2+; Calcium Orange; Calcofluor White; Carboxy-X-rhodarnine (5-ROX); Cascade Blue™; Cascade Yellow 399; Catecholarnine; CCF2 (GeneBlazer); CFDA; CFP-Cyan Fluorescent Protein; CFP/YFP; FRET; Chlorophyll; Chromomycin A; Chromomycin A; CL-NERF (Ratio Dye, pH); CMFDA; Coelenterazine; Coelenterazine cp (Ca2+Dye,); Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); CyQuant Cell Proliferation Assay; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiICl 8(5)); DIDS; Dihydorhodamine 123 (DHR); DiI (DiICl 8(3)); Dinitrophenol; DiO (DIOCl 8(3)); DiR; DiR (DiICl 8(7)); DM-NERF (high pH); DNP; Dopamine; DsRed; Red fluorescent protein; DTAF; DY-630-NHS; DY-635-NHS; EBFP; ECFP; EGFP; ELF 97; Eosin; Erytlrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyd Induced Fluorescence); FITC; FITC Antibody; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2, high calcium; Fura-2, low calcium; Fura-2/BCECF; Genacryl Brilliant Red B; Genacryl Brilliant Yellow 10GF; Genacryl Pink 3G; Genacryl Yellow SGF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; LIVE/DEAD Kit Animal Cells, Calcein/Ethidium homodimer, LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARFI; Sodium Green; Spectrum Aqua; Spectrum Green; Spectrum Orange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYT; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85;SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-CyS); TRITC; TetramethylRodaminelsoThioCyanate; True Blue; TruRed; Ultralite; Uranine B; Uvitex SFC; wt GFP; WW 781; X-Rhodarnine; XRITC; Xylene Orange; Y66F; Y66H; Y66W; Yellow GFP; YFP; YO-PRO-1; YO-PRO-3; YOYO-1 and YOYO-3.

Apart from the fluorescence-based detection protocols, which are used to discern the presence or pattern of a target molecule or molecules (such as an antigen) either spacitally (tissue distribution, localization, etc.) or phenotypically (expression levels, etc.), chromogen-based visualization protocols are also available, either on their own or in combination with fluorescence detection. Preferred chromogens include diaminobenzidine (DAB), but many other chromogens are also available. In some cases, staining is intensified by addition of a second factor such as heavy metal ions, including nickel and cobalt. One or more steps of detection procedures, such as staining (incubation) and color development are enhanced by exposing the sample to the irradiation disclosed herein. Commonly used chromogen substrate solutions include the following: DAB-Peroxidase Substrate Solution (Brown); DAB-Peroxidase Substrate Soluiton (Gray); DAB-Peroxidase Substrate Solution (Black); DAB-Peroxidase Substrate Solution (Blue); AEC-Peroxidase Substrate Solution (Red); BDHC-Peroxidase Substrate Solution (Blue); TMB-Peroxidase Substrate Solution (Blue); New Fuchsin Alkaline Phosphatase Substrate Sulution (Red); BCIP/NBT Alkaline Phosphatase Substrate Solution (Blue).

Where the present invention is used in conjunction with a technique or assay system that is immuno-affinity-based, the invention may facilitate any such process by promoting chemical reactions or molecular intaractions. Typical immuno-affinity reagents that are used in the art include: an antibody, an antigen-binding fragment thereof, and other engineered derivatives thereof, including so-called Affibody® molecules, all of which are discussed in further detail elsewhere herein. Furthermore, it should be appreciated that such immuno-affinity agents may be used for determining spatial distributions of a target molecule of interest (for example, localization of an antigen in a cell or tissue), as well as for compositional determination by measuring quantities or comparative levels of a target molecule of interest present in a sample (for example, immunoprecipitation or fluorometric assays).

As a more specific example of how the present invention is applied to facilitate existing methodology, the methods provided herein can be easily adapted for steps involved in identification, detection and/or measurement of a known biological marker or markers present in a sample. For example, microwave can be used in immunofluorescence technique, such as double immunocytochemical staining. It has been shown that moderate microwaving does not elute antibodies, but prevents their reactions with subsequently applied reagents.

Thus, using the methods presented herein, microwaving performed in between the first and second staining cycles permits improved double indirect immunofluorescence staining with antibodies raised in the same species. Moreover, microwaving also inhibits reactions with endogenous immunoglobulins present in extracellular compartments. This substantially reduces background in indirect immunostaining of mouse tissues with mouse monoclonal antibodies, for instance, and further enhance the results, as compared to those obtaind using a conventional microwave oven.

Diagnostic immunohistology (DIHC), therefore, is an essential discipline that provides the accurate identification of infectious organisms, distinction between morphologically-similar undifferentiated tumors, separation of benign and malignant neoplasms, and prognostication of malignancies. The technology often directly affects prognosis, selection of therapy, as well as patients' response to treatment. Therefore, improved methods for diagnostic immunohistology that allow faster, more accurate results are of much interest, and the methods provided herein embrace such improvement. Such methods may involve a variety of tissue types and cell types. For example, the methods of the present invention are useful for diagnostic, as well as prognostic processes of a disease or disorder, cancer in particular, involving tissues and/or cells including: nervous system, breast cancers, skin cancers, renal cell carcinoma, prostate cancer, lung cancers, gastronintestinal stromal tumor, bone lesions, nasal and paranasal sinus tumors, melanoma, hodgkin and non-hodgkin lymphomas, vascular neoplasms, uterus tumors, thyroid cancer, pleomorphic sarcomas, among others.

Commercially available reagents that may be used to determine cells and tissues of epithelial and/or endothelial origin include: CA19-9 antibody [241]; CD166 antibody [3A6] (FITC); CD166 antibody [L50]; Cytokeratin 13 antibody [IC7]; Cytokeratin 13 antibody [AE8]; Cytokeratin 13 antibody [KS-1A3]; Cytokeratin 13 antibody [KS-1A3]; Cytokeratin 4 antibody [6B10]; Cytokeratin 4 antibody [6B10]; D240 antibody [D2-40]; prediluted Differentiated Endothelial Cells antibody [1F10 ]; EBP50 antibody; EBP50 antibody [EBP-10]; Endothelial Cell antibody [BW-200 ]; Endothelial Cell antibody [PAL-E]; Endothelial Cell antibody [RECA-1]; Endothelium antibody [1.BB.803]; Endothelium antibody [EN4]; Endothelium antibody [MRC OX43] (FITC); Endothelium antibody [PAL-E]; EpCAM antibody [0.N.277]—BSA and Azide free; EpCAM antibody [AUA1]; EpCAM antibody [B29.1 (VU-ID9)]; EpCAM antibody [B29.1 (VU-ID9)] (FITC); EpCAM antibody [302 (323/A3)]; EpCAM antibody [B302 (323/A3)]; EpCAM antibody [Ber-EP4]; EpCAM antibody [E144]; EpCAM antibody [HEA125]; prediluted EpCAM antibody [VU-1D9]; EpCAM antibody [VU-1D9]; prediluted Filaggrin antibody; Filaggrin antibody [FLG01]; Filaggrin antibody [SPM181]; Filaggrin antibody [SPM181]; prediluted Filaggrin protein (Tagged); Gastric Carcinoma antibody [BY-1 (3H11)]; HMW Cytokeratin antibody [34bE12]; HMW Cytokeratin antibody [34betaE12]; prediluted HMW Cytokeratin antibody [DE-SQ]; Junctional Adhesion Molecule C antibody [CRAM-18 F26]; Junctional Adhesion Molecule C antibody [CRAM-19 H36]; Kallikrein 6 antibody; Kallikrein 6 antibody—Catalytic domain; Kallikrein 6 antibody—Kallikrein loop; Kallikrein 6 peptide (Catalytic domain); Kallikrein 6 peptide (Kallikrein loop); Mammaglobin antibody; Mammaglobin antibody; Mammaglobin antibody; Mesothelin antibody [K1]; Mesothelin antibody [SPM143]; Mesothelin antibody [SPM143]; prediluted MUC1 antibody [E29]; MUC1 antibody [LBS-1]; MUC1 antibody [LH39]; MUC1 antibody [PR4D1]; MUC1 antibody [VU-1D9]; PDZK1 antibody; Plakophilin 3 antibody [23E3/4]; Plakophilin 3antibody [E612B11F8]; Prostate Secretory Protein/PSP antibody [YPSP-1]; RECA1 antibody [HIS52]; SLC26A3 antibody—Azide free; TEM7 antibody [197C193]; TEM8 antibody; TEM8 antibody; TEM8 antibody [200C1339]; TEM8 peptide; TEM8 peptide (551-564); TEM8 peptide (92-107); THSD1 antibody [TX17.10]; THSD1 antibody [TX17.10] (Biotin); THSD1 antibody [TX17.10] (FITC); URO10 antibody [T43]; URO2 antibody [S2]; URO4 antibody [S27]; URO5 antibody [Ti 6]; URO7 antibody [S22]; URO8 antibody [F31]; URO9 antibody [Om5]; Urothelium antibody [LBS 8]; and Vascular Endothelium antibody [10].

Commercially available reagents for determinating cells and tissues of brain and neuronal origin or indicative of some of the neuronally derived diseases useful for use in the methods described herein include: 200 kDa+68 kDa Neurofilament antibody [SPM 145]; 200 kDa+68 kDa Neurofilarnent antibody [SPM145], prediluted; DYX1C1 antibody DYX1C1 peptide (408-420); AKAP9 antibody; AKAP9 antibody [17G10]; Arg 3.1 antibody; Arg 3.1 peptide Doublecortin (phospho S28) antibody—Neuronal Marker; Doublecortin antibody—Neuronal Marker; Doublecortin peptide Doublecortin peptide; Doublecortin peptide—phospho S28; Doublecortin peptide—phospho S297; DYX1C1 antibody; DYX1C1 peptide (408420); LXN antibody; LXN protein (T7 Tag); MAP1B antibody [3G5]—Neuronal Marker; MAP1B antibody [AA6]; MAP1B antibody [SPM283], prediluted; MAP2a+MAP2b antibody [AP20] (FITC); MAP2a+MAP2b antibody [AP20]—Neuronal Marker; MAP2a+MAP2b antibody [MT-01]—Neuronal Marker; MAP2a+MAP2b antibody [MT-07]; MAP2a+MAP2b antibody [SPM284], prediluted; neuron specific beta III Tubulin antibody; neuron specific beta III Tubulin antibody [TU-20]—Neuronal Marker; neuron specific beta III Tubulin antibody [TUJ-1]—Neuronal Marker; neuron specific beta III Tubulin peptide; PGP9.5 antibody; PGP9.5 antibody—Neuronal Marker; PGP9.5 antibody [10A1]—Neuronal Marker; PGP9.5 antibody [13C4/13C4]—Neuronal Marker; PGP9.5 antibody [13C4]; PGP9.5 antibody [31A3]; PGP9.5 peptide; PGP9.5 peptide (175-191); Spectrin (non erythroid) antibody [D8B7].

Commercially available tumor-associated reagents useful for use in the methods described herein include: ADAMTS1 antibody; ADAMTS1 antibody—Aminoterminal end; ADAMTS1 antibody—Carboxyterninal end; ADAMTS1 antibody—Propeptide domain; ADAMTS1 peptide (Aminoterminal end); AIB1 antibody [0.T.198]; AIB1 antibody [AX 15]; ALK antibody; ALK antibody [5A4]; ALK antibody [SP8]; ALK antibody [SP8], prediluted; ALK antibody, prediluted; ALK protein; alpha 1 Fetoprotein Receptor antibody [2B8]; alpha 1 Fetoprotein Receptor antibody [2B8] (HRP); alpha 1 Fetoprotein Receptor antibody [5E1]; alpha Lactalbumin antibody; alpha Lactalbumin antibody (Alkaline Phosphatase); alpha Lactalbumin antibody [0.N.14]; alpha Lactalbumin antibody [F20.16]; AMACR+p63 antibody [4A4 (p63)]—Cocktail of mouse monoclonal and rabbit polyclonal; AMACR antibody; AMACR antibody [13H4]; AMACR antibody, prediluted; Anti-ErbB 2 Affibody® Molecule Imaging Agent; Anti-ErbB2 Affibody® Molecule; Anti-ErbB2 Affibody® Molecule (Agarose); Anti-ErbB2 Affibody® Molecule (Biotin); Anti-ErbB2 Affibody® Molecule (FITC); Anti-ErbB2 Affibody® Molecule (HRP); Anti-HSA Affibody® Molecule; Anti-HSA Affibody® Molecule (Biotin); ASAP1/DDEF1 antibody; Axin 1 antibody; Axin 1 peptide (850-862); Ax1 antibody; Ax1 antibody; BCA225 antibody [CU18]; BCAR3 antibody; BCAR3 peptide; BCAS2 antibody; BCAS2 protein (T7 Tag); BCRP/ABCG2 antibody [BXP-21]—Hematopoietic/Neural Stem Cell Marker; BCRP/ABCG2 antibody [BXP-34]—Hematopoietic/Neural Stem Cell Marker; BCRP/ABCG2 antibody [BXP-53]; BCRP/ABCG2 antibody [BXP-9]; Benzopyrene antibody BRCAA1 antibody; c-Kit (phospho Y568+Y570) antibody; c-Kit (phospho Y703) antibody; c-Kit (phospho Y721) antibody; c-Kit (phospho Y730) antibody; c-Kit (phospho Y823) antibody; c-Kit (phospho Y936) antibody; c-Kit antibody; c-Kit antibody [104D2]; c-Kit antibody [104D2] (Allophycocyanin); c-Kit antibody [104D2] (Biotin); c-Kit antibody [104D2] (Phycoerythrin); c-Kit antibody [2B8]; c-Kit antibody [2B8] (Allophycocyanin/Cy5.5®); c-Kit antibody [2B8] (Biotin); c-Kit antibody [2B8] (Cy5) Cy5® conjugated; c-Kit antibody [2B8] (FITC); c-Kit antibody [2B8] (PE/Cy5) PE/Cy5® conjugated; c-Kit antibody [2B8] (Phycoerythrin); c-Kit antibody [B-K15]; c-Kit antibody [B-K15] (Biotin); c-Kit antibody [B-K15] (Phycoerythrin); c-Kit antibody [T595]; c-Kit antibody [Y145]; c-Kit antibody, prediluted; c-Kit peptide—phospho Y703 (phospho and non-phospho pair); c-Kit peptide—phospho Y721 (phospho and non-phospho pair); c-Kit peptide—phospho Y730 (phospho and non-phospho pair); c-Kit peptide—phospho Y823 (phospho and non-phospho pair); c-Kit peptide—phospho Y936 (phospho and non-phospho pair); CA125 antibody [10G12]; CA19-9 antibody [0.N.36]; CA19-9 antibody [192]; CA19-9 antibody [241]; CA19-9 antibody [BC/121SLE]; CA19-9 antibody [SPM110]; CA19-9 antibody [SPM110], prediluted; Carcino Embryonic Antigen CEA antibody; Carcino Embryonic Antigen CEA antibody [1C10] (HRP); Carcino Embryonic Antigen CEA antibody [1C11]; Carcino Embryonic Antigen CEA antibody [1C3]; Carcino Embryonic Antigen CEA antibody [1C7]; Carcino Embryonic Antigen CEA antibody [26/3/13]; Carcino Embryonic Antigen CEA antibody [26/5/1]; Carcino Embryonic Antigen CEA antibody [85A12]; Carcino Embryonic Antigen CEA antibody [C6G9]; Carcino Embryonic Antigen CEA antibody [CB30]; Carcino Embryonic Antigen CEA antibody [CI-P83-1] (FITC); Carcino Embryonic Antigen CEA antibody [CLB-139]; Carcino Embryonic Antigen CEA antibody (Col-1); Carcino Embryonic Antigen CEA antibody [Col-1], prediluted; Carcino Embryonic Antigen CEA antibody [II-7]; Carcino Embryonic Antigen CEA antibody [NCRC16 (AKA 161)]; Carcino Embryonic Antigen CEA antibody, prediluted; Carcino Embryonic Antigen CEA antibody, prediluted; Carcino Embryonic Antigen CEA protein; Carcino Embryonic Antigen CEA protein Carcino Embryonic Antigen CEA protein; Carcino Embryonic Antigen CEA protein (Mitogen Free); Cathepsin P antibody; CCK4 antibody; CD15 antibody [0.N.79]; CD15 antibody [28]; Dimethylbenzanthracene antibody; DLC1 antibody; Dysadherin antibody; Dysadherin peptide (164-178); EMAP II antibody; EMAP II antibody [546-2]; EpCAM antibody [0.N.277]—BSA and Azide free; EpCAM antibody [AUA1]; EpCAM antibody [B29.1 (VU-ID9)]; EpCAM antibody [B29.1 (VU-ID9)] (FITC); EpCAM antibody [B302 (323/A3)]; EpCAM antibody [Ber-EP4]; EpCAM antibody [E144]; EpCAM antibody [HEA125], prediluted; EpCAM antibody [VU-1D9]; EpCAM antibody [VU-1D9], prediluted; ErbB 2 (phospho T686) antibody; ErbB 2 (phospho Y1221+Y1222) antibody; ErbB 2 (phospho Y1221) antibody; ErbB 2 (phospho Y1222) antibody; ErbB 2 (phospho Y1248) antibody; ErbB 2 (phospho Y1248) antibody [PN2A]; ErbB 2 (phospho Y877) antibody; ErbB 2 antibody; ErbB 2 antibody—BSA and Azide free; ErbB 2 antibody [10C7]; ErbB 2 antibody [24D2]; ErbB 2 antibody [24D2] (Allophycocyanin); ErbB 2 antibody [24D2] (FITC); ErbB 2 antibody [24D2] (Phycoerythrin); ErbB 2 antibody [3B5]; ErbB 2 antibody [9G6]; ErbB 2 antibody [CB11]; ErbB 2 antibody [CB11], prediluted; ErbB 2 antibody [ICR12]; ErbB 2 antibody [ICR52]; ErbB 2 antibody [ICR55]; ErbB 2 antibody [N24]; ErbB 2 antibody [SP3]; ErbB 2 antibody [SPM172], prediluted; ErbB 2 antibody [V2]; ErbB 2 antibody [V2W]; ErbB 2 antibody, prediluted; ErbB 2 peptide—phospho Y1248; ErbB 3 antibody; ErbB 3 antibody [RTJ2];ErbB 3 antibody [SGP1]; ErbB 4 antibody [HFR1]; ErbB2 peptide; EWSR1 antibody; Factor XIIIa antibody; Factor XIIIa antibody [AC-1A1]; Factor XIIIa antibody [AC-1A1], prediluted; FLJ23603 antibody; Folate Binding Protein antibody; Folate Binding Protein antibody (Biotin); Folate Binding Protein antibody (HRP); Folate Binding Protein antibody (HRP)—Azide free; Folate Binding Protein antibody [LK26]; Gastric Carcinoma antibody [BY-1 (3H11)]; GCDFP 15 antibody; GCDFP 15 antibody. [0.N.307]; GCDFP 15 antibody [23A3]; GCDFP 15 antibody [23A3], prediluted; GCDFP 15 antibody [3G153]; GCDFP 15 antibody [D6], prediluted; GCDFP 15 antibody [SPM135]; GCDFP 15 antibody [SPM135], prediluted; GCDFP 15 peptide; GCDFP 15 protein; GPCR GPR124 antibody; GSTM1+GSTM2 antibody; GSTM1+GSTM2 peptide (207-217); GTAM12 antibody [B43.12]; HAS3 antibody; HAS3 peptide; HE4 antibody; Hepatocellular carcinoma antibody [CHALV1]; HHV8 Cyclin antibody [23]; Hippostatin antibody; HL60 antibody [IPO-M6]; HL60 Whole Cell Lysate; HPV18 E6 antibody [289-13965]; HPV18 E6 antibody [BF7]; Human c-Kit ELISA Kit—1×96 Well Plate; Human c-Kit ELISA Kit—2×96 Well Plates; Human colorectal adenocarcinoma antibody [C241:5:1:4]; Human Prostate Tumor antibody [YPMA-2]; Human Serum Albumin antibody [OCH1E5]; Human Serum Albumin antibody [OCH1E5], prediluted; Human sVCAM1 ELISA Kit—1×96 Well Plate; Human VCAMI ELISA Kit—2×96 Well Plates; IF127 antibody; INSM1 antibody; IRS1 (phospho S307) antibody; IRS1 (phospho S312) antibody; IRS1 (phospho S616) antibody; IRS1 (phospho S636) antibody; IRS 1 (phospho S639) antibody; IRS 1 (phospho Y 1179) antibody; IRS1 (phospho Y1229) antibody; IRS1 (phospho Y612) antibody; IRS1 (phospho Y869) antibody; IRSI (phospho Y896) antibody [EP260Y]; IRS1 (phospho Y941) antibody; IRS1 antibody; Kalinin antibody [GB3]; Kallikrein 14 antibody; Kallikrein 14 antibody—Catalytic domain; Kallikrein 14 antibody—Kallikrein loop; Kallikrein 14 peptide; Kallikrein 14 peptide (Catalytic domain); Kidney tumor (human): renal cell carcinoma tissue slides; Kidney tumor (human): Wilm's tumor tissue slides; Kindlin antibody; LIMA/SIMA antibody [4A1]; Liver tumor (human): hepatocellular carcinoma tissue slides; LSM1 antibody; LSM1 protein (T7 Tag); Lung Carcinoma antibody [CHLG-26]; Lung Carcinoma antibody [T2101]; Lung Carcinoma antibody [TFS-4]; MA2 antibody; MAGE 1 antibody; MAGE 1 antibody [E22-11B2-E9]; MAGE 1 antibody [SPM282]; MAGE 1 antibody, prediluted; MAGEA3 antibody; MAGEA6 antibody; MAGEA8 antibody; MAGED2 antibody; Mammaglobin antibody; Marek's Disease antibody; MASPIN antibody; MASPRN antibody—Aminoterminal end; MASPIN antibody—Helix C-D; MASPIN antibody—RCL Shoulder; MASPIN peptide; MASPIN peptide (Aminoterminal end); MASPIN peptide (Helix C-D); MASPIN peptide (RCL Shoulder); MCSP antibody [LHM 2]; MelanA antibody; MelanA antibody [A103]; MelanA antibody [DT101+BC199]; MelanA antibody [M2-7C10+M2-9E3]; MelanA antibody [M2-7C10]; MelanA antibody, prediluted; Melanocyte cell surface antigen antibody [Mel.2]; Melanoma antibody [HMB45+DT101+BC199+T311]; Melanoma antibody [HMB45(IgG1/k)+M2-7C10 (IgG2b)+M2-9E3 (IgG2b)+T311 (IgG2a)]; Melanoma antibody [HMB45+DT101+BC199]; Melanoma antibody [HMB45]; Melanoma antibody [HMB45], prediluted; Melanoma antibody [LHM 3]; Melanoma antibody [PAL-M2]; Melanoma antibody [PNL2]; Melanoma gp100antibody; Melanoma gp100antibody [SPM142]; Melanoma gp100 antibody [SPM142], prediluted; Melanoma gp100 antibody, prediluted; Melanoma gp100 protein; Membralin antibody; Mesothelin antibody [K1]; Mesothelin antibody [SPM143]; Mesothelin antibody [SPM143], prediluted; Mesothelioma antibody [HBME1]; MTGR1 antibody; MUC1 antibody; N myc interactor antibody; N ras+c Ha ras antibody; NABC1 antibody; NAPSIN A antibody; NAPSIN A peptide (408-421); Nck alpha antibody; Nck beta antibody; Nck beta antibody, prediluted; NSP 5 alpha 3 alpha antibody; Ovarian Carcinoma-associated Antigen antibody [OV632]; Ovarian Carcinoma-associated Antigen OA3 antibody [OVTL-16]; p15 INK4b antibody; p15INK4b antibody—BSA and Azide free; p15 INK4b antibody [15P06]; p15 INK4b antibody [DCS-114]; pan CEACAM antibody [D14HD11]; pan CEACAM antibody [TET2]; pan Mucin antibody [b12]; PHAP3 antibody; PHAP3 peptide; PRAME antibody; PRAME peptide; Prostate Secretory Protein/PSP antibody [YPSP-1; Prostatic Acid Phosphatase antibody; Prostatic Acid Phosphatase antibody [4LJ]; Prostatic Acid Phosphatase antibody [PASE/4LJ], prediluted; Prostatic Acid Phosphatase antibody [SPM312], prediluted; Prostatic Acid Phosphatase antibody, prediluted; PRUNE antibody; PSA antibody; PSA antibody [2H9]; PSA antibody [5A6] (HRP); PSA antibody [5G6]; PSA antibody [8A6]; PSA antibody [A67-B/E3]; PSA antibody (ER-PR8]; PSA antibody [ER-PR8], prediluted; PSA antibody [PS1] (HRP); PSA antibody [PS6]; PSA antibody [PS6] (HRP); PSA antibody [PSA1]; PSA antibody, prediluted; PSA peptide (244-254); PSCA antibody; PSCA antibody, prediluted; PSGR antibody; Psoriasin antibody [47C1068]; Psoriasin antibody [47C1068]-Azide free; PTP4A3antibody; PTP4A3antibody [02301]; PTP4A3 antibody [111AT714]; PTP4A3 peptide (5-18); PTP4A3 protein (T7 Tag); Renal Cell Carcinoma (gp200) antibody [MG38]; Renal Cell Carcinoma (gp200) antibody [MG38] (FITC); Renal Cell Carcinoma (gp200) antibody [MG38] (Phycoerythrin); Renal Cell Carcinoma (gp200) antibody [PN-15]; Renal Cell Carcinoma (gp200) antibody [PN-15], prediluted; Renal Cell Carcinoma (gp200) antibody [RC38]; Renal Cell Carcinoma (gp200) antibody [SPM314], prediluted; Reticulon IA antibody [MON160]; Reticulon IA antibody [MON 161]; Reticulon IA antibody [MON162]; Rituximab antibody [MB2 A4]; Rituximab antibody [MB2 A4] (FITC); Secretory Component Glycoprotein antibody [0.N.556]; Secretory Component Glycoprotein antibody [SC-05]; Secretory Component Glycoprotein antibody [SPM217]; SSX2IP antibody; SSX2IP peptide (602-614); STEAP antibody; Stefin A antibody; Stefin A antibody, prediluted; TAG72antibody [0.N.561]-BSA and Azide free; TAG72 antibody [0.N.562]—BSA and Azide free; TAG72 antibody [B72.3]; TAG72antibody [B72.3], prediluted; TAG72antibody [CC49], prediluted; TAG72 antibody [CC49]; TAG72 antibody [SPM148]; TAG72antibody [SPM148], prediluted; TCR alpha+beta+epithelial tumor antibody [R73+CC52]; TEM7 antibody [197C193]; TEM8antibody; TEM8antibody [200C1339 ]; TEM8peptide; TEM8 peptide (551-564); TEM8 peptide (92-107); TFEB antibody; Thomsen-Friedenreich Antigen antibody [A78-G/A7]; Thomsen-Friedenreich Antigen antibody [SPM320], prediluted; TM4SF3 antibody; TMEM16A antibody; TPD52L1 antibody [d1C5]; TPD52L1 protein (His tag); TRP1 antibody [Ta99]; TRP1 antibody [Ta99], prediluted; Tyrosinase antibody; Tyrosinase antibody (Alkaline Phosphatase); Tyrosinase antibody [T311]; Tyrosinase antibody, prediluted; Tyrosinase Related Protein 75 antibody [3F388]; Tyrosinase Related Protein 75 antibody [Ta99]; UBIAD1 antibody; URO10 antibody [T43]; URO2 antibody [S2]; URO4 antibody [S27]; URO5 antibody [T16]; URO7 antibody [S22]; URO8 antibody [F31]; URO9 antibody [Om5]; Uroplakin III antibody; VIP Receptor I antibody; VIP Receptor I antibody [AS58]; VPAC2 antibody; VPAC2 antibody [AS69]; WHSC1/NSD2 antibody; Wilms Tumor Protein antibody; Wilms Tumor Protein antibody [WLM04]; Wilms Tumor Protein antibody, prediluted; XAGE1 antibody; XTP4 antibody; YB1 antibody; YB1 peptide; ZAP70 (phospho Y292) antibody; ZAP70 (phospho Y315+Y319) antibody; ZAP70 (phospho Y319) antibody; ZAP70 (phospho Y319) antibody [E227]; ZAP70 (phospho Y493) antibody; ZAP70 antibody; ZAP70 antibody [1E7.2]; ZAP70 antibody [1E7.2] (Phycoerythrin); ZAP70 antibody [E267]; ZAP70 antibody [SB70] (Alkaline Phosphatase); ZAP70 antibody [SB70] (Biotin); ZAP70 antibody [SB70] (HRP); ZAP70 antibody [SBZAP]; ZAP70 antibody [SBZAP] (Alkaline Phosphatase); ZAP70 antibody [SBZAP] Biotin); ZAP70 antibody [SBZAP] (FITC); ZAP70 antibody [SBZAP] (Phycoerythrin); ZAP70 antibody [YE291]; ZAP70 antibody [ZAP-03]; ZAP70 antibody, prediluted; ZNFNIA2 antibody;14-3-3 zeta antibody; 14-3-3 zeta antibody [8C3]; and 14-3-3 zeta antibody, prediluted.

There are many other useful markers and reagents that may be helpful for determining cell types, developmental stages, embryonic origins, lineages, differentiation status, disease progression, and so on.

An exemplary procedure of specimen staining used in a typical evaluation scenario for pathology is provided below: (1) After the sample(s) are fixed, (2) embedded in paraffin, (3) cut in 8 to 10 micron-thick sections, (4) and placed on a glass microscope slide. (5) One slide is stained with H&E (hematoxylin and eosin) for review by a pathologist, (6) who makes a preliminary diagnosis. (7) If there is need of more histological information to differentiate the origin of the lesion (tumor) (as from lymphoid, epithelial, nervous, adipose or mesenchymal, etc.), (8) a panel of differentiating antibodies are applied to tissue sections, (9) processed appropriately for label detection, and (10) presented to pathologist for review. For example, to identify epithelial origin, CD45 leukocyte common antigen, cytokeratin and Epithelial membrane antigen reagents are commonly used. To identify mesenchymal, vimentin is commonly used. To identify neural tissues, s-100 reagent is commonly used. To identify cycling tumors (prognostic for some tumors), ki-67 is commonly used. To identify breast cancer, reagents specific for estrogen or progesterone receptor is commonly used. To identify prostate cancer, prostate specific antigen is used. To identify thyroid or lung carcinoma, TTFI is used. The samples may be subjected to irradiation as decribed herein during the steps (1), (4), (5), (8) and/or (9). Microwave irradiation during washing steps of (8) and (9), for instance, may markedly reduce non-specific binding of antibodies, thereby enhancing specific signals.

According to the present disclosure, methods of improving chemical process that comprise an intermolecular interaction are provided.

As used herein, the term “intermolecular interaction” shall encompass interactions characterized by a covalent bonding or non-covalent bonding, and shall include interactions that occur within a molecule, as well as interactions that occur between two or more molecules. Examples of interactions that occur within a molecule include, but are not limited to: an interaction between two domains of a protein and a palindromic interaction of a nucleic acid molecule. Non-covalent interactions may occur between two molecules of the same class or two molecules of different classes. The former includes, for example, an interaction between two polypeptides (i.e., protein-protein interactions) and an interaction between two complementary nucleic acid fragments. The latter includes an interaction between a protein and a nucleic acid, an interaction between a protein and a lipid, and so on.

According to the invention, intermolecular interactions include covalent intermolecular interactions. Covalent bonding is a description of chemical bonding that is characterized by the sharing of pairs of electrons between atoms. In short, attraction-to-repulsion stability that forms between atoms when they share electrons is known as covalent bonding. Intermolecular interactions of the invention also include non-covalent intermolecular interactions. Noncovalent bonding refers to a variety of interactions that are not covalent in nature between molecules or parts of molecules that provide force to hold the molecules or parts of molecules together, usually in a specific orientation or conformation. These noncovalent interactions include: ionic bonds, hydrophobic interactions, hydrogen bonds, Van der Waals forces (aka London dispersion forces), and Dipole-dipole bonds. As used herein, “non-covalent bonding,” “non-covalent interactions,” and “non-covalent forces” all refer to these forces as a whole without specifying or distinguishing which specific forces are involved: noncovalent interactions often involve several of these forces working in concert. Noncovalent bonds are weak by nature and must generally therefore work together to have a significant effect.

In the context of proteins, intramolecular noncovalent interactions are largely responsible for the secondary and tertiary structure of proteins and therefore the protein's function in the mechanisms of life. Moreover, intermolecular noncovalent interactions are responsible for protein complexes (quaternary structure) where two or more proteins function in a coherent mechanism. Therefore, electromagnetic fields provided in the invention disclosed herein may provide dielectric energy sufficient to directly or indirectly alter one or more aspects of these interactions of biomolecules including proteins and nucleic acids.

More specifically, some embodiments of the invention are based on the premise that under certain conditions described elsewhere herein dielectric energy may “drive away”molecules in a solution such that the molecules, an antibody for instance, may be directed toward a target, an antigen, for instance, thereby speeding up the reaction process significantly. Additional advantage based on the technology is that in some cases it may eliminate the need for mechanical agitation of samples during incubation, for example, allowing users to carry out assays using a significantly reduced amount of reagents, and yet be able to obtain a comparable result. Without intending to be limiting, apart from direct thermal effects generated by irradiation, vortex currents (i.e., local microfluidic circulation) resulting from the electromagnetic energy field may act to enhance local mixing of unbound reagents, enhancing their interaction with the substrate tissue/epitopes/cells. Accordingly, these methods may generate a higher yield of detectable signals and lower background in a shorter period of time, and at a lower cost.

In some embodiments, the invention relates to techniques that utilize hybridization of nucleic acids. Thus, methods are provided in which one or more steps of performing such techniques are improved by the irradiation means of the present invention. Hybridization means for DNA or RNA to pair by hydrogen bonds to a complementary sequence, forming a double-stranded polynucleotide. The term is often used to describe the binding (or annealing) of a DNA probe, or the binding (or annealing) of a primer to a DNA strand during a polymerase chain reaction (PCR). The term is also often used to describe the reformation (renaturation) of complementary strands that were separated by thermal denaturation. Hybridization of nucleic acids is used in a variety of assays and screenings and can take place in vitro, in situ or in vivo.

In the context of histochemical applications, perhaps the most common technique employing hybridization of nucleic acids is in situ hybridization, as well as its variations. In situ hybridization (ISH) is a type of hybridization technique that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). DNA in situ hybridization can be used to determine the structure of chromosomes. Fluorescent DNA in situ hybridization (FISH) can, for example, be used in medical diagnostics to assess chromosomal integrity. RNA in situ hybridization (hybridization histochemistry) is used to measure and localize mRNAs and other transcripts within tissue sections or whole mounts. For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e. only exact sequence matches will remain bound). Then, the probe that was labeled with either radio-, fluorescent- or antigen-labeled bases (e.g. dixoygenin) is localized and quantitated in the tissue using either autoradiography, fluorescence microscopy or immunohistochemistry, respectively. These techniques can also use two or more probes, labeled with radioactivity or the other non-radioactive labels, to simultaneously detect two or more transcripts. Thus, the present invention is applicable to any of the foregoing variations of the technique. While the exact mechanisms underling the effect of microwave irradiation on hybridization of nucleic acid are not entirely understood, it is widely accepted and adapted in routine laboratory practice that microwave irradiation exerts desirable effects. That is, microwave treatment can often replace protenase K digestion for frozen sections; enhance protenase K digestion in paraffin sections; denature mRNA structure to enable better probe access; preserve tissue and cell architecture; and inactivate endogenous alkaline phosphatase within sections to reduce background when immunohistochemistry-based probe detection is used. For example, in fluorescence in situ hybridization (FISH), signals are enhanced by microwave pulses applied during the DNA-DNA hybridization process, particularly for a single/low-copy probe. Similarly, using microwave irradiation, it is possible to repeatedly carry out microwave-assisted fluorescence in situ hybridization. The ability to perform re-hybridization is valuable, particularly for pathology archive sections, for instance, or any other cases where samples are available only in limited quantities or expensive. For example, the methods of the instant invention may be adapted for protocol involving stripping the probe from the pathology archive sections with HCl and re-hybridizing with the next probe by intermittent microwave irradiation. In addition, these methods may be easily adapted by a skilled artisan for use in high throughput screening involving nucleic acid hybridization.

The invention is useful for histochemical applications involving a wide range of biological samples, and may be especially suited for use in processing fixed samples. As used herein, “a fixed sample” is a sample that has been treated with a suitable fixative for preservation. A number of fixatives are commonly used and are discussed elsewhere. For instance, in some preferred embodiments, the sample may be dehydrated and/or paraffin-embedded. These histological techniques are well known in the art. In some cases, samples require antigen retrieval procedure to uncover antigenic sites from tissue sections. An exemplary schematic is provided in FIG. 7 (Act 400). Commonly used methods are either microwave heat treatment using a conventional microwave oven by boiling the sections in 0.01M citrate buffer (e.g., pH 6.0) for 10-20 minutes or enzyme digestion by incubating sections with a proteolytic enzyme (such as trypsin (0.05% (v/v) in PBS with 0.1 % CaCl₂) at 37° C., or at room temperature for 10-20 minutes. Therefore, the irradiation apparatus of the present invention can be used in lieu of a standard microwave oven and will be able to produce superior results. Those skilled in the art can determine the conditions of concentration, time and temperature without undue experimentation. Thus, the methods disclosed herein can replace most if not all of these methods and produce superior results.

In further embodiments, the invention is useful for shortening the processing time of samples for scanning electron microscopy. To demonstrate, microwave irradiation can be applied for processing microorganisms, such as flagellated bacteria. In the simplest methodology, the bacteria are placed on a cover glass, air-dried, and submitted to conductivity stain (such as 10 ml of 5% carbolic acid solution, 2 g of tannic acid, and 10 ml of saturated aluminum sulfate, and H₂O). Alternatively, the samples may be double-fixed (glutaraldehyde and then osmium, for instance), submitted to conductivity stain, dehydrated with ethanol, treated with hexamethyldisilazine (HMDS), and dried at 35° C. for 5 minutes. In either protocol, the steps from fixation to treatment with HMDS is carried out under microwave irradiation for 2 minutes in an ice bath. Either of the techniques provides fast methods and still preserves the morphology of the bacterial samples adequately.

In other embodiments, the biological sample may be a frozen sample. The frozen sample may be either previously fixed (such as formalin-fixed) or flash-frozen without chemical fixation. For example, flash-frozen food samples, such as produce, may be screened for possible contamination, such as bacteria and chemical toxins (insecticides, etc.). Yet in certain circumstances, a freshly dissociated samples (i.e., harvested freshly) may be used. For example, under certain circumstances, it may be advantageous to harvest and process a tissue or cell, for instance during a surgery, for obtaining immediate results. Thus, the invention makes it possible to carry out in-surgery, i.e., real-time analyses of biological samples.

The invention further includes these methods that are used for chemical and histochemical analyses of a living cell or cells. Depending on the cell type or growth state, as well as other factors, cells may be in suspension; alternatively, cells may be adhered to an appropriate substrate, such as a culture dish or coated glass slide or cover slip, among others. Thus, the methods disclosed herein are suitable for biological samples which are immobilized or mounted, as well as for biological samples which are present in solution.

In some embodiments, methods are provided, whereby the present invention is used to determine a genotype and/or phenotype of a biological sample. Typically, these methods involve subjecting the biological sample to the electromagnetic field described herein to enhance the subsequent genotypic and/or phenotypic characterization of the sample.

As used herein, the term “genotype” refers to the specific genetic make-up of an individual, in the form of DNA, i.g., alleles. An example to illustrate genotype is the single nucleotide polymorphism or SNP. An SNP occurs when corresponding sequences of DNA from different individuals differ at one DNA base, for example where the sequence AAGCCTA changes to AAGCTTA. This contains two alleles: C and T. SNPs typically have three genotypes, denoted generically AA Aa and aa. In the example above, the three genotypes would be CC, CT and TT. Other types of genetic marker, such as microsatellites, can have more than two alleles, and thus many different genotypes. In contrast, the “phenotype” of an individual cell or organism, depending on the context, is either its total moephological or physical appearance and constitution or a specific manifestation of a trait, such as cell type-specific features, or in a case of an individual organism, size, eye color, or behavior that varies between individuals. Phenotype is determined to a large extent by genotype, or by the identity of the alleles that an individual carries at one or more positions on the chromosomes. Many phenotypes are determined by multiple genes and influenced by environmental factors. These genetic association studies can be used to determine the genetic risk factors associated with a disease. It may also be possible to differentiate between populations (both at the cellular and systematic levels) who may or may not respond favorably to a particular drug treatment. Such an approach is often referred to as personalized medicine or pharmacogenetics, and the present invention finds applications in improving many possible steps of genotypic and phenotypic determinations.

For example, in some embodiments, one or more steps of genotypic or phenotypic determination using the methods decribed herein involve detecting at least one biomarker.

Biomarker (also “bio-marker”) is defined as a substance used as an indicator of a biologic state. It may be an indication of different things in different contexts, and non-limiting examples are shown below.

In some cases, a bio-marker can be any kind of molecule indicating the existence (past or present) of living organisms. In particular, in the fields of geology and astrobiology biomarkers are also known as biosignatures. The term is also used to describe biological involvement in the generation of petroleum. The methods according to the present invention may, therefore, provide a more rapid, sensitive means of detecting and identifying biosignatures, as compared to conventional methods.

In biology and medicine, a biomarker can be a substance whose detection indicates a particular disease state or risk thereof (for example, the presence of a particular antibody may indicate an infection). More specifically, a “biomarker” indicates a change in expression or state of a protein that correlates with the risk or progression of a disease, or with the susceptibility of the disease to a given treatment. Once a proposed biomarker has been validated, its monitoring can be used to diagnose disease risk, presence of disease in an individual, or to tailor treatments for the disease in an individual (choices of drug treatment or administration regimes). Examples include, but are not limited to, many cancer-specific or tumor-specific antigens and viral proteins (such as HIV envelope protein). More specifically, and without intending to be limiting, cancer-specific markers that are commonly used include, inter alia, CEA, CA19-9, CA125, NY-ESO-1, MAGE-A1, MAGE-A2, MAGE-A3, MAGE-A4, MAGE-A6, MAGE-A9 MAGE-A10, MAGE-A12, MAGE-C2, BAGE, GAGE, GnTV, HERV-K-MEL, KK-LC-1, KM-HN-1, LAGE, mucin, NA-88, SAGE, Sp17, SSX2, SSX-4, TRP-2/INT-2.

In cell biology in particular, biomarkers include cell-specific molecules that allow for the detection and isolation of a particular cell type. For example, PSA, beta-HCG-(Human chorionic gonadotropin), AFP-(Alpha-fetoprotein), AFP-L3-(a lectin-reactive AFP) and Thyroglobulin all represent some of the known tissue-specific proteins. For example, if man has an elevated PSA, a search for prostate cancer will be undertaken. If an individual has an elevated level of beta-HCG, AFP or AFP-L3%, a search for a testicular or liver cancer, respectively, will be made. In another example, Oct-4 is used as a biomarker to identify embryonic stem cells. Similarly, there are numerous proteins whose expression is restricted to certain tissue or organ, such as the nervous system, blood vessels, cardiac tissue, smooth muscles, endothelial tissue, etc. Furthermore, a biomarker can also be used to indicate exposure to various environmental substances in epidemiology and toxicology. In these cases, the biomarker may be the external substance itself (e.g. asbestos particles or NNK from tobacco), or a variant of the external substance processed by the body (e.g., a metabolite). In genetics, a biomarker (identified as genetic marker) is a fragment of DNA sequence that causes disease or is associated with susceptibility to disease. Biomarkers may also be indicated by resistence to certain drugs, such as antibiotics. Accordingly, the present invention can be used in conjunction with a number of techniques that are available in the art to accelerate or enhance the process of detection and/or analysis based on any of these biomarkers, inter alia.

For example, using the apparatus and methods of the invention, it is possible to improve simultaneous imaging of a cellular antigen and the corresponding chromosomal locus in samples, including pathology archives. A mutistep procedure for genotype-phenotype analysis involves microwave-assisted fluorescence in situ hybridization combined with immunofluorescence in the same cell. Microwave irradiation can be employed for steps of fixation of a sample; pre-treatment of the sample prior to FISH or CISH for antigen retrieval (typically ∞10 minutes); each washing; probe incubation, etc. Essentially, any such protocols that are published and typically used for the technique can be adapted for better results using the present invention.

Similarly, the invention allows improved in situ PCR methods for detecting nucleic acids of low abundance. For example, detection of transferred foreign genes in histological sections, for example, has been challenging due to low transfection efficiency and a low copy number of vectors present in the sample. In these cases, localization of transferred vectors can be sufficiently achieved by using microwave irradiation, as described herein, during fixation and/or during proteinase K digestion.

Yet another application of the irradiation methods of the invention is for detecting chromosomal abnormality, e.g., centromere numerical abnormality, using microwave-assisted FISH in various clinicopathological settings. Using intermittent microwave irradiation, multiple probes can be effectively employed. Because the methods provided herein can enhance specificity of signals and at the same time can reduce non-specific background, while speeding up each incubation and waching step involved, superior results can be obtained, as compared to those obtained by using a standard microwave oven.

A unique advantage made possible by the present invention is that the methods provided herein comprising one or more steps of analyzing a biological sample and determining a phenotype of the biological sample may be performed rapidly, in some cases in a matter of seconds to minutes, as opposed to hours to days. This offers a benefit, particularly during a surgery or in an emergency situation, where time is crucial.

It should be understood that the aspect of the invention drawn to a variety of chemical methods is not limited to use in a particular set of biological samples, but is widely applicable to any biological samples. Non-limiting examples include: a tissue sample, a bodily fluid sample, a biopsy sample, a cell sample, a blood sample, a serum sample, a plasma sample, a urine sample, a hair sample, an airborne sample and a food sample. Any of the foregoing biological samples may be collected and used for the methods described herein for purposes of: clinical studies, pathological analyses, diagnosis of a disease or disorder, prognosis of a disease or disorder, treatment of a disease or disorder, histological analyses, morphological analyses, genetic analyses, public health (contamination analyses for food and water, bio-defense, epidemiological analyses), and so on.

Based on the nature of laboratory and clinical techniques, as well as the type of analytical or medical instruments of choice, to which the present invention is to be applied, those skilled in the art can implement the technology to achieve improved results. Accordingly, the methods provided herein may be useful in accelerating or enhancing the process of identification, diagnosis and/or prognosis of a disease, disorder, and other medical conditions including but not limited to: Allergy; Aspergillosis; B 19 parvovirus; Bacterial infections; Blastomycosis; various Cancers; Candidiasis; Cardiomyopathy; Coccidioidomycosis; Cryptococcus; Cryptosporidiosis; Cytomegalovirus (CMV); Depression; Diabetes; Entamoeba histolytica; Giardia lamblia; Gingivitis; Guillain-Barré syndrome; Gynaecomastia (breast enlargement); Hairy leukoplakia; Hepatitis A; Hepatitis B; Hepatitis C; Herpes simplex; Histoplasmosis; HIV-associated dementia; HIV-associated salivary disease; Hodgkin's disease; Human herpes virus 6; Human papilloma virus; Isosporiasis; Kaposi's sarcoma; Lactic acidosis/acidaemia; Leishmaniasis; Lung cancer; Lymphocytic interstitial pneumonitis; Malaria; Microsporidiosis; Menopause; Spontaneous Miscarriage; Molluscum contagiosum; Multicentric Castleman's disease; Mycobacterium avium intracellulare (MAI); Mycobacterium haemophilum; Mycobacterium kansasii; Neuropathy; Neutropenia; Non-Hodgkin's lymphoma; Osteonecrosis; Osteoporosis; Pancreatitis; Pelvic inflammatory disease; Penicilliosis; Persistent generalised lymphadenopathy; Pneumocystis pneumonia (PCP); Pregnancy; Progressive multifocal leukoencephalopathy (PML); Psoriasis; Pulmonary arterial hypertension; Q fever; Renal (kidney) disease; Salmonellosis; Schistosomiasis and other worm and fluke infections; Seborrhoeic dermatitis; Syphilis; Testicular cancer; Testosterone deficiency; Thrombocytopenia; Thrombotic thrombocytopenic purpura; Tinea; Toxoplasmosis; Tuberculosis; Ulcers; Vacuolar myelopathy; Varicella zoster virus and Wasting syndrome.

According to another aspect of the invention, methods for accelerating or enhancing a wide variety of binding assays, are provided. Typically, embodiments drawn to binding assays comprise several steps: (1) obtaining a test sample; (2) mixing together the test sample with a target compound so as to allow them to come into contact; and, (3) subjecting the test mixture containing the test sample and the target compound to an electromagnetic field localized to the immediate vicinity of the test mixture; and finally, (4) detecting bindings between the test sample and the target compound. As described in more detail above, the electromagnetic field can provide a level of power and a duration of time sufficient to achieve acceleration or enhancement of the binding assay so as to produce overall improvement in the assay system.

As used herein, the term “binding assay” is intended to include, not only assays that examine the level of interaction between at least two molecules by detecting complex formation, but also screening assays that are based on binding between molecules. An array of libraries are available with which such screening may be performed. The term, “a test sample” refers to a molecule or a pool of molecules, defined or undefined, which are to be tested for its ability to selectively interact (ie., bind) with a given molecule or compound of choice, which is referred here as “a target compound” and works as a “capture agent.” Preferably, a target compound is a defined compound. Each of the two counterparts (a test sample and a target compound) may consist of a number of different classes of molecules or agents, such as polypeptides, nucleic acids, small molecules (such as hormones, groth factors, cytokines, chemokines, and various other ligands etc.), lipids, carbohydrates, synthetic materials, and so on. The invention in this aspect is not limited for use in certain classes of molecules, but rather, the invention is widely applicable to situations, where an assay is to be performed and interaction (or binding) between such molecules is to be detected.

While applicable to an array of biological, biochemical and analytical assays that utilize binding or association between a molecule or molecules, the invention is particularly suited for a variety of immunoassays, including many variations thereof. Some examples of such embodiments are dicussed below.

As used herein, the term “immunoassay” shall encompass a large variations of immuno-affinity-based biochemical tests that measure the level of a substance in a biological sample, using the binding of an antigen to an antibody, antibodies, fragments thereof or engineered derivatives (e.g., Affibody® molecules) there of. These assays take advantage of the specific affinity of an antibody to its antigen. Monoclonal antibodies are often used as they only usually bind to one site of a particular molecule, and therefore provide a more specific and accurate test, which is less easily confused by the presence of other molecules. However, polyclonal antibodies may be also used for the immunoassays described herein. Both the presence of antigen or antibodies can be measured. For instance, when detecting infection the presence of antibody against the pathogen is measured. For measuring hormones such as insulin, the insulin acts as the antigen. In certain cases, use of smaller, engineered derivatives of immuno-affinity reagents, such as Affibody® molecules, in lieu of or in combination with an antibody or antibodies, is perferred.

Antibodies are well known to those of ordinary skill in the science of immunology. As used herein, the term “antibody” means not only intact antibody molecules but also fragments of antibody molecules retaining binding ability. Such fragments are also well known in the art and are regularly employed both in vitro and in vivo. In particular, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J NucL. Med. 24:316-325 (1983)). Preferably the immunoglobulin is selected from the following Ig isotypes: IgA, IgM, IgD, IgE and IgG (IgG comprises four sub-classes based on differences in the H chains, i.e. IgG1, IgG2, IgG3 and IgG4).

According to one embodiment, the antibody is an intact soluble monoclonal antibody. An intact soluble monoclonal antibody, as is well known in the art, is an assembly of polypeptide chains linked by disulfide bridges. Two principle polypeptide chains, referred to as the light chain and heavy chain, make up all major structural classes (isotypes) of antibody. Both heavy chains and light chains are further divided into subregions referred to as variable regions and constant regions. As used herein the term “monoclonal antibody” refers to a homogenous population of immunoglobulins which specifically bind to an epitope (i.e. antigenic determinant).

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fe region has been enzymatically cleaved, or which has been produced without the Fe region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation. The terms Fab, Fe, pFc′, F(ab′)₂ and Fv are used consistently with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

Therefore, antibodies of the invention may be single chain antibodies or may be single domain antibodies (intrabodies or intracellular antibodies). Intrabodies are generally known in the art as single chain Fv fragments with domains of the immunoglobulin heavy (VH) and light chains (VL). Well-known functionally active antibody fragments include but are not limited to F(ab′)₂, Fab, Fv and Fd fragments of antibodies. These fragments which lack the Fe fragment of intact antibody, clear more rapidly from the circulation, and may have less non-specific tissue binding than an intact antibody (Wahl et al., J Nucl. Med. 24:316-325 (1983)). For example, single-chain antibodies can be constructed in accordance with the methods described in U.S. Pat. No. 4,946,778 to Ladner et al. Such single-chain antibodies include the variable regions of the light and heavy chains joined by a flexible linker moiety. Methods for obtaining a single domain antibody (“Fd”) which comprises an isolated variable heavy chain single domain, also have been reported (see, for example, Ward et al., Nature 341:644-646 (1989), disclosing a method of screening to identify an antibody heavy chain variable region (VH single domain antibody) with sufficient affinity for its target epitope to bind thereto in isolated form). Methods for making recombinant Fv fragments based on known antibody heavy chain and light chain variable region sequences are known in the art and have been described, eg., Moore et al., U.S. Pat. No. 4,462,334. Other references describing the use and generation of antibody fragments include e.g., Fab fragments (Tijssen, Practice and Theory of Enzyme Immunoassays (Elsevieer, Amsterdam, 1985)), Fv fragments (Hochrnan et al., Biochemistry 12: 1130 (1973); Sharon et al., Biochemistry 15: 1591 (1976); Ehrilch et al., U.S. Pat. No. 4,355,023) and portions of antibody molecules (Audilore-Hargreaves, U.S. Pat. No. 4,470,925). Thus, those skilled in the art may construct antibody fragments from various portions of intact antibodies without destroying the specificity of the antibodies for their target.

As is well-known in the art, the complementarity determining regions (CDRs) of an antibody are the portions of the antibody which are largely responsible for antibody specificity. The CDRs directly interact with the epitope of the antigen. In both the heavy chain and the light chain variable regions of IgG immunoglobulins, there are four framework regions (FR1 through FR4) separated respectively by three complementarity determining regions (CDR1 through CDR3). The framework regions (FRs) maintain the tertiary structure of the paratope, which is the portion of the antibody which is involved in the interaction with the antigen. The CDRs, and in particular the CDR3 regions, and more particularly the heavy chain CDR3 contribute to antibody specificity. Because these CDR regions and in particular the CDR3 region confer antigen specificity on the antibody these regions may be incorporated into other antibodies or peptides to confer the identical specificity onto that antibody or molecule.

Detecting the quantity of antibody or antigen can be achieved by a variety of methods which the art is familiar with. One of the most common is to label either the antigen or antibody. The label may consist of an enzyme (ie., enzyme immunoassay, or EIA), radioisotopes such as 1-125 Radioimmunoassay (RIA) or fluorescence. Other techniques include agglutination, nephelometry, turbidimetry and Western Blot, or immunoblot. Chemical coupling of such a label or labels to a suitable reagent (such as an antibody) may also be enhanced by the present invention, as schematically shown in FIG. 7 (Act 500).

Furthermore, immunoassays can be competitive or noncompetitive, and can be homogeneous or heterogeneous. In a competitive immunoassay, the antigen in the unknown sample competes with labeled antigen to bind with antibodies. The amount of labeled antigen bound to the antibody site is then measured. In this method, the response will be inversely proportional to the concentration of antigen in the unknown. This is because the greater the response, the less antigen in the unknown was available to compete with the labeled antigen.

In noncompetitive immunoassays, also referred to as the “sandwich assay,” antigen in the unknown is bound to the antibody site, then labeled antibody is bound to the antigen. The amount of labeled antibody on the site is then measured. Unlike the competitive method, the results of the noncompetitive method will be directly proportional to the concentration of the antigen. This is because labeled antibody will not bind if the antigen is not present in the unknown sample.

A heterogeneous immunoassay may require an extra step to remove unbound antibody or antigen from the site, usually using a solid phase reagent. Immunoassays have a particularly important role in the diagnosis of a number of medical conditions, diseases and disorders. Non-limiting examles include the diagnostic applications of the following: viral infections (HIV, HPV, HVC, HVB, etc.), bacterial infections (Staphylococcus aureus; ‘Gram negative’ bacteria; methicillin-resistant S. aureus (MRSA); Shigella; Campylobacterjejuni; Salmonella; Clostridium; Clostridium difficile; Listeria; Salmonella; Campylobacter; Lymphogranuloma venereum (LGV); Streptococcus pneumoniae; Haemophilus influenzae; Pseudomonas aeruginosa; Rhodococcus equi, Nocardia; Bordetella; Bartonella; Staphylococcus; Mycobacterium avium intracellulare (MAI); Pseudomonas; Neisseria gonorrhoeas, etc.), various cancers, blood disorders, liver disorders, kidney disorders, skin disorders, allergies, etc. In addition, the invention is useful when combined with routinely used techniques to determine or monitor medical conditions such as pregnancy, miscarriage, menopause, diabetes, and so on.

In further embodiments, therefore, invention is implemented to improve ELISA assays, including variations thereof.

The Enzyme-Linked ImmunoSorbent Assay, or ELISA, is well known in the art. ELISA is a biochemical technique used mainly in immunology to detect the presence of an antibody or an antigen in a sample. Generally, it uses two antibodies: one antibody is specific to the antigen; and the other reacts to antigen-antibody complexes, and is coupled to an enzyme. This second antibody, which accounts for “enzyme-linked” in the test's name, can also cause a chromogenic or fluorogenic substrate to produce a signal. Because the ELISA can be performed to evaluate either the presence of antigen or the presence of antibody in a sample, it is a useful tool both for determining serum antibody concentrations (such as with the Human Immunodeficiency Virus, HIV test or West Nile Virus) and also for detecting the presence of antigen. It has also found applications in the food industry in detecting potential food allergens, such as milk, peanuts, walnuts, almonds, and eggs.

The steps of a typical or “indirect” ELISA for determining serum antibody concentrations may comprise the following: (1) Apply a sample of known antigen to a surface, often the well of a microtiter plate. The antigen is fixed to the surface to render it immobile; (2) The plate wells or other surface are then coated with serum samples of unknown antibody concentration, usually diluted in another species' serum. The use of non-human serum prevents non-specific antibodies in the patient's blood from binding to the antigen; (3) The plate is washed, so that unbound antibody is removed. After this wash, only the antibody-antigen complexes remain attached to the well; (4) The second antibodies, which will bind to any antigen-antibody complexes, are added to the wells. These second antibodies are coupled to the substrate-modifying enzyme; (5) Wash the plate, so that excess unbound antibodies are removed; (6) Apply a substrate which is converted by the enzyme to elicit a chromogenic or fluorescent signal; and (7) View/quantify the result using a spectrophotometer or other optical device. Recently, a conventional microwave oven has been adapted for ELISA protocols to accelerate the process. In these cases, typically ˜2.45-GHz microwave irradiation was found to be effective. However, problems persisted due to uneven heating of within a sample or across samples, added to the fact that conventional microwave ovens lack sufficient fine tuning of wavelength, power, the range of irradiation. In view of the foregoing, therefore, the technology of this invention brings about a number of superior features. Using the above ELISA protocol as an example, the irradiation means of the present invention may be applied to one or more of the steps (1), (2), (3), (4), (5) and (6), to obtain rapid, often superior results.

ELISA may be run in a qualitative or quantitative format. Qualitative results provide a simple positive or negative result for a sample. In certain circumstances, this is a prefered mode of detection. These include, for example, certain blood tests (Rh+/−; A, B, AB, OO), screening for infections (HIV, Hepatitis, etc), and pregnancy test. The cutoff between positive and negative is determined by the analyst and may be statistical. In some cases, two or three times the standard deviation is may be used to distinguish positive and negative samples. In quantitative ELISA, the optical density or fluorescent units of the sample is interpolated into a standard curve, which is typically a serial dilution of the target. Enhanced sensitivity of the assay based on the implementation of the methods described herein may reduce the number of samples (e.g., dilutions) and may also reduce the amount (volume) of the reagensts necessary for each sample. Because of even spatial distribution of irradiation across a target surface, deviations across samples are expected to be significantly reduced.

Many modified ELISA assays are used. In a so-called “sandwich ELISA” protocol, for example, the following steps are typically involved: (1) Plate is coated with a capture antibody; (2) sample is added, and any antigen present binds to capture antibody; (3) detecting antibody is added, and binds to antigen; (4) enzyme-linked secondary antibody is added, and binds to detecting antibody; (5) substrate is added, and is converted by enzyme to detectable form.

A less-common variant of the “sandwich” ELISA technique, is used to detect sample antigen. The steps are as follows: (1) Prepare a surface to which a known quantity of antibody is bound; (2) Apply the antigen-containing sample to the plate; (3) Wash the plate, so that unbound antigen is removed; (4) Apply the enzyme-linked antibodies which are also specific to the antigen; (5) Wash the plate, so that the unbound antibodies are removed; (6) Apply a chemical which is converted by the enzyme into a fluorescent signal; and (7) View and analyze the result: fluoresce signal means that the sample contained antigen.

The image to the right, includes an additional step, the addition of ‘detecting antibody’, used to avoid the expensive conjugation process that would be necessary to create enzyme-linked antibodies for every antigen one might want to detect. By using an enzyme-linked antibody that binds the Fc region of other antibodies, this same enzyme-linked antibody can be used in a variety of situations.

Competitive ELISA is a third use of ELISA, which is based on competitive binding. The basic steps for this ELISA may include: (1) Unlabeled antibody is incubated in the presence of its antigen; (2) These bound antibody/antigen complexes are then added to an antigen coated well; (3) The plate is washed, so that unbound antibody is removed. (The more antigen in the sample, the less antibody will be able to bind to the antigen in the well, hence “competition.”); (4) The secondary antibody, specific to the primary antibody is added. This second antibody is coupled to the enzyme; and (5) A substrate is added, and remaining enzymes elicit a chromogenic or fluorescent signal. Thus, for competitive ELISA, the higher the original antigen concentration, the weaker the eventual signal. Any of the above steps that involve incubations of antibody or antibodies, binding reaction (complex formation) as well as washing steps can benefit from the electromagnetic irradiation of the present invention.

One widely used clinical application based on the principle of ELISA is ELISPOT. The Enzyme-linked immunosorbent spot (ELISPOT is a common method for monitoring immune responses in humans and animals. The ELISPOT assay is based on, and was developed from a modified version of the ELISA immunoassay. ELISPOT assays were originally developed to enumerate B cells secreting antigen-specific antibodies, and have subsequently been adapted for various tasks, especially the identification and enumeration of cytokine-producing cells at the single cell level. Simply put, at appropriate conditions the ELISPOT assay allows visualization of the secretory product of individual activated or responding cells. Each spot that develops in the assay represents a single reactive cell. Thus, the ELISPOT assay provides both qualitative (type of immune protein) and quantitative (number of responding cells) information, and may be improved in sensitivity by implimenting the apparatus, methods and uses disclosed herein.

Yet another example of application of the invention is the secretion assay. Secretion assay is a process used in cell biology to identify cells that are secreting a particular peptide (often a cytokine). Usually, a cell that is secreting the protein of interest is isolated using an antibody-antibody complex that coats the cell and is able to “catch” the secreted molecules. And this capture step may be greatly facilitated by subjecting the sample to electromagnetic irradiation. For example, reaction time for the capture step may be effectively reduced by 30%, 40%, 50%, 60%, 70%, 80%, 90% or more. In parallel, sensitivity of the assay may be enhanced by up to 300%. The cell is then detected by another fluorochrome-labelled antibody, and is subsequently extracted using a process called fluorescent-activated cell sorting (FACS). The detection step (schematically shown in FIG. 7, Act 710), as well as the sorting step (schematically shown in FIG. 7, Act 700), both of which involve binding of specific labels (schematically shown in FIG. 7, Act 600 & 610), may also be be accelerated by the use of the invention. The FACS method is broadly similar to the ELISA antibody format, except that the encapsulated cells remain intact. This is advantageous as the cells are still living after the extraction has taken place.

A number of commercial applications exist for secretion assay. One such example is the Gel Microdrop (GMD) techonology, developed by One Cell Systems. One Cell asserts that GMD typically recovers a higher number of viable secreting cells than other methods, whilst ignoring any cells which are not secreting the desired protein.

In further embodiments, the invention may be effectively implemented for use in techniques and instruments that exploit nano-particles, such as fluorescent or magnetic nano-particles. It is, for example, possible to extract the secreting cells using a magnetic-based separation system or using a flow cytometer. In other applications fluorescent nanoparticles are used as a dye conjugated to antibodies used to identify or decorate epitopes of interest. In certain embodiments, the invention is used to accelerate or enhance assays that are aimed to determine relative affinity between two molecules or compounds. Generally speaking, the term “affinity” denotes preferential interaction between such molecules or compounds. Relative affinity may be assayed based on either binding constant or dissociation constant. In either case, exposing such a sample mixture to certain levels of irradiation during a step comprising complex formation or dissociation, for instance, at a frequency in the range of 10 megahertz, delivered at 100 vpp, may heighten the sensitivity of the assay and reduce the reaction time and reaction volume. Those skilled in the art will thus be able to use the invention disclosed herein to perform such assays and obtain superior results at a lower cost.

As would be clear to those skilled in the art, interactions between molecules, or compounds in the context of the aspect of the invention extend beyond protein-prtein interaction, but further include interactions involving nucleic acid hybridization. “Nucleic acids” as used herein include DNA, RNA, analogs thereof, combination thereof and mixture thereof. For example, “DNA” may be a fragment of genomic DNA, cDNA, plasmid DNA, oligonucleotides, and so on. Similarly, RNA may include, inter alia, mRNA and siRNA. A “substrate” in these cases, may take a variety of forms: for example, nucleic acid samples may be disposed onto a microchip (gene chip, etc.), may be contained in a microtube or well, may be coupled to the surface of such substrates, or in some cases may be in a solution. Samples may be provided as isolated samples, crude samples, extracts, and may be purified as is or may be present in a cell or in situ. In addition, nucleic acids may be obtained from a cell, tissue, or viral source; alternatively, nucleic sampels may be chemically synthesized. These technologies are well known in the art.

For most of the related applications, the interaction between a test sample and a target compound involving nucleic acids represents annealing, i.e., hybridization of complementary base pairs, for example, DNA:DNA, DNA:RNA, and RNA:RNA. However, the use of the invention in enhancing nucleic acid interactions further embraces interaction between a nucleic acid molecule and a second molecule/compound of a different kind, particularly polypeptides. Thus, in certain embodiments, the invention provides methods for accelerating for enhancing interactions between an aptamer and its target compond.

A “target compound”, again, may constitute a wide range of molecules. Aptamers are oligonucleic acid or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. More specifically, aptamers can be classified as: DNA or RNA aptamers and peptide aptamers. The former consist of (usually short) strands of oligonucleotides. And the latter consist of a short variable peptide domain, attached at both ends to a protein scaffold. Each is descrived in further details below.

RNA and DNA aptamers are nucleic acid species that have been evolutionary engineered through in vitro selection or equivalently, SELEX (systematic evolution of ligands by exponential enrichment) to bind to various molecular targets such as small molecules, proteins, nucleic acids, and even cells, tissues and organisms. Aptamers offer the utility for biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein which have good solubility and compacity properties. Currently, the bacterial protein Thioredoxin-A is the most used scaffold protein, the variable loop being inserted within the reducing active site, which is a -Cys-Gly-Pro-Cys- loop in the wild protein, the two Cysteins lateral chains being able to form a disulfide bridge. Peptide aptamer selection can be made using different systems, but the most used is currently the yeast two-hybrid system. Selection of Ligand Regulated Peptide Aptamers (LiRPAs) has been demonstrated.

As used herein, a biomolecule refers to a chemical compound that naturally occurs in living organisms, fragments thereof, and synthetic analogs and derivatives thereof. Biomolecules consist primarily of carbon and hydrogen, along with in some cases, nitrogen, oxygen, phosphorus and sulfur. Other elements sometimes are incorporated but are much less common. A diverse range of biomolecules exist, including: Small molecules (Lipid, Phospholipid, Glycolipid, Sterol, Vitamin, Hormone, Neurotransmitter, etc.); Carbohydrate (Monosaccharide, Disaccharide, Polysaccharide, etc.); Monomers (Amino acid, Nucleotide, Phosphate, Monosaccharide, etc.); Polymers (Peptide, Oligopeptide, Polypeptide, Protein, Nucleic acid, i.e. DNA, RNA, Oligosaccharide, Polysaccharide, etc.); Macromolecules (Prion, etc.). In some cases, biomolecules include modified and/or non-natural amino acids, modified and/or nucleic acid analogues (such as GNA, PNA, TNA, LNA and morpholino). Accordingly, biomolecules include: a hormone, a neurotransmitter, a cytokine, a chemokine or a growth factor, and functional analogues thereof; as well as an agonist, an antagonist, a ligand, an inhibitor, a blocker and a co-factor.

In further embodiments, the invention contemplates that the test sample comprises a small molecule.

As used herein, “a small molecule” includes both naturally occurring small molecules and synthetic small molecules. These, and other compounds, may be used to examine selective or preferential binding to a candidate molecule. Thus, combining screening technologies and assay systems that are available in the art, the present invention may greatly accelerate the overall process of such assays. In some embodiments, binding assays are used to test binding/interactings of two more defined molecules. Yet in other embodiments, assays may use a known/defined molecule as a target compound, and screen for candidate molecule or molecules that exhibit selective binding.

Some small molecules are hormones or analogues thereof. Non-limiting examples of such molecules that may be used for purposes of screening or binding assays of the invention include: Melatonin (N-acetyl-5-methoxytryptamine); Serotonin; Thyroxine (thyroid hormone); Triiodothyronine (thyroid hormone); Epinephrine (or adrenaline); Norepinephrine (or noradrenaline); Dopamine; Antimullerian hormone (or mullerian inhibiting factor or hormone); Adiponectin; Adrenocorticotropic hormone (or corticotropin); Angiotensinogen and angiotensin; Antidiuretic hormone (or vasopressin, arginine vasopressin); Atrial-natriuretic peptide (or atriopeptin); Calcitonin; Cholecystokinin; Corticotropin-releasing hormone; Erythropoietin; Follicle-stimulating hormone; Gastrin; Ghrelin; Glucagon; Gonadotropin-releasing hormone; Growth hormone-releasing hormone; Human chorionic gonadotropin; Human placental lactogen; Growth hormone; Inhibin; Insulin; tyrosine kinase; Insulin-like growth factor (or somatomedin); Leptin; Luteinizing hormone; Melanocyte stimulating hormone; Oxytocin; Parathyroid hormone; Prolactin; Relaxin; Secretin; Somatostatin; Thrombopoietin; Thyroid-stimulating hormone; Thyrotropin-releasing hormone; Cortisol; Aldosterone; Testosterone; Dehydroepiandrosterone; Androstenedione; Dihydrotestosterone; Estradiol; Estrone; Estriol; Progesterone; Calcitriol (Vitamin D3); Prostaglandins; Leukotrienes; Prostacyclin and Thromboxane

Similarly, exemplary ligands include, but are not limited to: 5-hydroxytryptamine, acetylcholine, adenosine, noradrenaline, adrenaline, anaphylatoxin C5a, C5a des Arg74, anaphylatoxin C3a, angiotensin, apelin, neuromedin B, gastrin-releasing peptide, bradykinin, cannabinoid, CXCL1, CXCL2, CXCL3, CXCL4, CXCL5, CXCL6, CXCL7, CXCL8, CXCL9, CXCL10, CXCL11 (eotaxin), CXCL12, CXCL13, CXCL14, CXCL15, CXCL16, macrophage derived lectin, CCL1, CCL2, CCL3, CCL4, CCL5 (RANTES), CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27; CCL28, CX3CL1, XCL1, XCL2, cholecystokinin, gastrin, dopamine, endothelin 1, endothelin 2, endothelin 3, long chain carboxylic acids, carboxylic acids, acetate, bile acids, galanin, motilin, ghrelin, thyroid-stimulating hormone, luteinizing hormone, chorionic gonadotropin, follicle-stimulating hormone, gonadotrophin-releasing hormone, histamine, KiSS-1 gene product, leukotriene D4, leukotriene C4, leukotriene B4, 5-oxo-ETE, lipoxin A, lysophosphatidic acid, sphingosine 1-phosphate, melanin-concentrating hormone, a-melanocyte stimulating hormone, adrenocorticotropic hormone, g-melanocyte stimulating hormone, b-melanocyte stimulating hormone, melatonin, neuromedin U. neuropeptide FF, Neuropeptide S, neuropeptide W, neuropeptide B, neuropeptide Y, pancreatic polypeptide, neurotensin, -N-formyl-L-Met-L-Leu-L-Phe (fMLP), nicotinic acid (low affinity), nicotinic acid (high affinity), b-endorphin, dynorphin A, b-endorphin, nociceptin/orphanin FQ, orexin A, orexin B, ADP, UTP, ATP, UDP, UDP-glucose, RF-amide P518 gene product, platelet-activating factor, prokineticins 1, prokineticins 2, prolactin-releasing peptide, prostaglandin D2, prostaglandin E2, prostaglandin F2a, prostacyclin, thromboxane A2, 11-dehydro-thromboxane B2, thrombin, serine proteases, relaxin, relaxin-3, INSL5, relaxin-3, somatostatin, (lyso)phospholipid mediators, substance P, neurokinin A, neurokinin B, b-phenylethylamine, tyramine, thyrotropin-releasing hormone, urotensin II, oxytocin, vasopressin, sphingosine 1-phosphate, neuropeptide head activator, lysophosphatidic acid, succinate, a-ketoglutarate, b-alanine, BAM8-22, cortistatin, RARRES2, resolvin E1, TIG2, estrogen, obestatin and oleoylethanolarnide. Cytokines include, without limitation, interleukin (IL)-1, IL-2, IL-3, IL4, IL-5, IL-6, IL-7, IL-8, IL-10, IL-12, IL-15, IL-18, interferon (IL)-α, IFN-β, IFN-γ, transforming growth factor (TGF)-β, tumor necrosis factor (TNF)-α, TNF-β, and granulocyte-macrophage colony-stimulating factor (GM-CSF). Immune cells may also upregulate certain molecules on their cell surface upon activation, for example, MHC class I, MHC Class II, CD11b, CD20, CD25, CD28, CD40, CD43, CD54, CD62L, CD69, CD71, CD80, CD86, CD95L, CD106, CD134, and CD134L. growth factors, such as platelet-derived growth factor, platelet factor 4, transforming growth factor-β; tissue factor VIIa, thrombin, fibrin, plasminogen-activator initiator, adenosine diphosphate, etc.

Yet other known compounds which may be commonly used include but are not limited to: A23187, Actinomycin D, AG 1295, AG 1478 HCl, Agmatine, Alamethicin, Albendazole, Aldosterone, Alsterpaullone, Amantadine HCl, Amiloride HCl, Aminopyridine, 4-Amiodarone HCl, Amodiaquine, Anandamide, Angiotensin II, Anisomycin, Anthopleurin C, Antimycin A3, Apamin, Arachidonic Acid, Artemisinin, Artemisinin, ATP, ATX II, Aurintricarboxylic Acid, Bafilomycin A1, Baicalein, BAPTA, Barium, Bcl-x(L) BH4(4-23), Benzamil HCl, Bepridil HCl, Berberine, Hemisulfate, Bromo-cAMP Sodium salt, 8-, Bromo-cGMP Sodium salt, 8-Bumetanide, Bungarotoxin A, Butyric Acid Na, C6 Ceramide, Caffeine, Calcitriol, Calcium, Calphostin C, Calyculin A, CaM kinase II, CAM Kinase II inhibitor, cAMP, Carbachol, Cerulenin, Charybdotoxin, Chlorophenylthio)-cAMP, 8-(4-, Chlorotoxin, Chlorpropamide, Clofilium Tosylate, Coichicine, Cyclopiazonic acid, Cyclosporin A, Cytochalasin B, D60, potassium, Damnacanthal, Dantrolene sodium salt, Daphnetin, Dapsone, dB-cAMP, Diethylamine NONOate/AM, Ditrazine, Dopamine, Doxorubicin HCl, Emodin, Epothilone A. Epothilone B, Erbstatin analog, Flecainide, Flufenamic Acid, Forskolin, Fura-2, Furosemide, Gadolinium, Galanthamine HBr, Geldanamycin, Genistein, GF-109203X HCl, Gingerol, Glibenclamide, Glimepiride, Glipizide, Go 6976, Guanosine, H-7 diHCl, HA-1077 diHCl, HA 14-1, Helenalin, HELSS, Heparin, Herbimycin A, Hymenialdisine, Hypericin, IAA-94 R(+)-, Indirubin-3, InsP3, Ionomycin Calcium salt, Isoproterenol, HCl, Ivermectin, KN-93, Lappaconitine HBr, Lavendustin A, Licochalcone-A, Synthetic, Linopirdine, Loperamide HCl, Mannoheptulose, Melatonin, Merocyanine 540, Methysergide, Mibefradil, Mosapride, N-Acetyl-L-cistern, N-Butyl-DNJ, HCl, Nateglinide, NEM, Nifedipine, Nitrendipine, NPY, NS 1619, NSC-65346, Ochratoxin A, Okadaic acid, Okadaic acid sodium, Ouabain, Pentamidine, Phenylarsine Oxide, Phorbol 12-myristate 13-acetate, Pinitol, Praziquantel, Propafenone HCl, Puromycin, Purvalanol A, Quercetin, Quinine, QX-222, Retigabine, Ryanodine, S-AMPA, S, 216763, SB 415286, SNAP, Sodium Azide, Staurosporine, Sumatriptan, Suramin, Taxol, Testosterone, Tetraethylammonium, Tetrodotoxin, Thapsigargin, Thiabendazole, Tinidazole, Tolazamide, Tolbutamide, TPEN, Triamterene, U-37883A, Valinomycin, Verapamil HCl, Veratridine, Vinblastine, Vitamin C, Xylazine, Z-VAD, 12(S)-HET, 2-APB, 4-Chloro-m-cresol, 6-Bnz-cAMP Na, 7-Nitroindazole, A23187, Adenophostin A, ADMB, Adrenaline, A, 1295, Aldosterone, Alsterpaullone, Aminoadipic Acid L-a-, Aminoguanidine, Hemisulfate, AMITU, Anandamide, Anhydroryanodine, Anisomycin, Apigenin, Arachidonic Acid, ATP, Aurintricarboxylic Acid, Baicalein, BAPTA AM, Bastadin 5, Berbamine, Bohemine, Bombesin Free Base, Bradykinin, Bromo-cAMP Sodium salt, 8-, Bromo-cAMP, 8-, Bromo-cGMP Sodium salt, 8-; Bromocriptine Mesylate, Butyric Acid, Na, C6 Ceramide, Caffeine; Calmidazoliu; chloride, Calmodulin, Calphostin C, CaM kinase II, CaM kinase II (290-309), CAM Kinase II inhibitor, CAM Kinase II selective substrate, CAM Kinase II substrate, CAM kinase IV substrate, cAMP, Cardiotoxin, CCCP, Chelerythrine chloride, Chenodeoxycholic Acid, Chlorophenylthio)-cAMP, 8-(4-, Chlorpromazine, Cholera toxin, Cholera toxin B subunit, Cilostamide, Compound 48/80, Curcumin, Cyclic ADPR, Cyclosporin A, Cyproterone acetate, Cytochalasin B, Cytochalasin D, D-erythro-Sphingosine-1-phosphate, D-IP3, Damnacanthal, Daphnetin, dB-cAMP, Diacylglycerol kinase inhibitor I, Diacylglycerol kinase inhibitor II, Diethylamine NONOate/AM, Diphenyleneiodonium chloride, Dipropyl-7-methylxanthine, 1,3-, Dipyridamole, Dopamine, Emodin, Enniatin B, Erbstatin analog, Etazolate HCl, ETPI, FFT, Fluoxymesterone, Fluphenazine N-mustard diHCI, Flutamide, Forskolin, Forskolin, 1,9-Dideoxy-, Forskolin, 6B-[B-(Piperidino)propionyl]-, HCl, Forskolin, 7B-Deacetyl-7B[a-(morpholino) butyryl]-, HCl, Furanophostin, Geldanamycin, Genistein, GF-109203X HCl, Go 6976, GSK-3, Guanosine, Guanylin, H-7 diHCl, H-8 diHCl, H-89, H-9 diHCl, HA-1004 HCl, HA-1077 diHCl, Haloperidol HCl, Helodermin, Heparin, Hepoxilin A3, Herbimycin A, Hymenialdisine, Hypericin, IBMX, Imperatoxin A, Indirubin-3, Ingenol, Inositol 1,4,5-trisphosphate, D-myo-, Inositol 1,4,6-trisphosphorothioate, L-chiro-, InsP3 lodotubercidin, 5-, IP4, Isoproterenol, HCl, Isoquinolinediol, 1,5-, Kemptide, Kenpaullone, Ketamine HCl, KN-62, KN-93, KT5720, L-cis-Diltiazem HCl, L-NAME HCl, L-NIL, DIHCI, L-NIO, L-NMMA, L-NNA, L-Thiocitrulline, 2HCl, Lavendustin A, Lithium Chloride, LY-294,002 HCl, LY-83,583, Lysophosphatidylcholine, L-alpha-, Mastoparan, Melatonin, Melittin, ML-7, ML-9, Molsidomine, Monomethyl-D-arginine acetate, NG-, Mycophenolic Acid, NAADP, NADPH, NG-Hydroxy-L-arginine Acetate Salt, Niflumic Acid, Nitro-D-arginine methyl ester, NG-HCl, NPC-15437 diHCl, NSC-65346, ODQ, Oleic Acid, Olomoucine, Ophiobolin A, PACOCF3, Papaverine HCl, Pentamidine, Pentoxifylline, Pertussis Toxin B oligomer, Phenoxybenzamine HCl, Phenylarsine Oxide, Phloretin, Phorbol 12,13-diacetate, Phorbol 12,13-dibutyrate, Phorbol 12-myristate 13-acetate, PIP2, PKA substrate, PKC selective substrate, PKC substrate, PPM-18, Progesterone, Protein kinase C selective inhibitor, Purvalanol A, Purvalanol B, Quercetin, Resiniferatoxin, Ribophostin, Ro 20-1724, Ro 31-8220, Rolipram, Rolipram, (R)-(−)-, Rp-cAMP TEA, Ru360, Ruthenium Red, Ryanodine, S-Benzylisothiourea HCl, SB 203580, SB 216763, SB 415286, SC-10, SC68376, SIN-1 chloride, SMT, SNAP, SNVP, SP 600125, Sp-cAMPS Triethylamine, Sp-cGMPS Triethylamine, SPC, Spermine NONOate, Spironolactone, SQ 22536, Staurosporine, Tamoxifen citrate, Taxol, Testosterone, Theophylline, TMB-8 HCl, Trifluoroperazine, TRIM, Vanadate, Vinpocetine, W-13 HCl, W-, HCl, W-7 HCl, Wortmannin, Xestospongin C, Xestospongin D, Xylazine, YC-1, Zaprinast, Zinc, Zinc protoporphyrin I, AACOCF3, Ammodytoxin, Antiflammin-1, Antiflammin-2, Aristolochic acid, Aurintricarboxylic Acid, Bombesin, Free Base, Bungarotoxin, B, C6 Ceramide, Compound 48/80, D-erythro-Sphingosine-1-phosphate, D609 potassium, Gelsolin, Heloderrnin, HELSS, Herbimycin A, Isoquinolinediol, 1,5-, MAFP, Manoalide, Mastoparan, Melittin, MJ33, NEM Neomycin Sulfate, Notexin Np, PACOCF3, Pertussis Toxin B oligomer, Phloretin, Phospholipase A2, Phospholipase D, PIP2, Propranolol HCl (+−), Protopine HCl, Quercetin, Suramin, Trifluoroperazine, U-73122, Wortmanni, 2-APB, Adenophostin A, Agatoxin IVA, w-, Agatoxin TK, w-, Aminoadipic Acid, L-a-, Antimycin A3, Bastadin 5, Bepridil HCl, BHQ, Bombesin, Free Base, C6 Ceramide, Calmidazolium chloride, Calyculin A, Cantharidic acid, Cerulenin, Chlorpromazine, Clozapine, Cyclopiazonic acid, Cyclosporin A, Cytochalasin B, DCCD, Digitonin, Diphenyleneiodonium chloride, DMHV, Endothall, Etomoxir, FCCP, Fostriecin, Furanophostin, L-SPD, Luciferin-Luciferase, Mannoheptulose, Melatonin, Microcystin LR, Microcystin-LF, N-Acetyl-L-cysteine, NADPH, Nodularin, Okadaic acid Okadaic acid sodium, Oligomycin, Phenylarsine Oxide, Pinitol, Potassium Cyanide, Rotenone, S-15176, Sodium Azide, Suramin, Tacrolimus, Tautomycin, Thapsigargin, Trifluoroperazine, Vanadate.

In certain embodiments, the test sample comprises a biosimilar. As used herein, “a biosimilar” is defined as a biopharmaceutical product, e.g., a drug with a protein as an active ingredient which is produced by genetically modified cell lines, having therapeutic equivalence as compared to original product but a small change in the manufacturing process results in an important impact on the efficacy and safety of a product.

In some embodiments, the target compound is immobilized on supports (ie., substrates), such as microtiter plates or beads, using procedures known to the artisan of ordinary skill in the art. These may take many forms, as deemed suited, for instance, microchip (DNA gene chip, etc.), dot blots, tissue blots, and others. Detection and analytical methods may also vary, as would be clear to those skilled in the art.

As used herein, “a high throughput assay” or “a high throughput screen” (HTS) refers to a highly parallel, partially or fully automated screening or assaying system designed to systematically process a large number of samples for specific biological activity of interest. It is sometimes also referred to as “a high throughput screening.” Generally, a high throughput screen uses robotics to simultaneously test thousands of distinct compounds in functional and/or binding assays. Therefore, such screening is often used to look for drug candidates.

Through a combination of modem robotics, data processing and control software, liquid handling devices, and sensitive detectors, HTS allows a researcher to effectively conduct millions of biochemical, genetic or pharmacological tests in a short period of time. Through this process one can rapidly identify active compounds, antibodies or genes which modulate a particular biomolecular pathway. The results of these experiments provide starting points for drug design and for understanding the interaction or role of a particular biochemical process in biology. In essence, HTS uses a brute-force approach to collect a large amount of experimental data—usually observations about how some biological entity reacts to exposure to various chemical compounds—in a relatively short time. A screen, in this context, is the larger experiment, with a single goal (usually testing a scientific hypothesis), to which all this data may subsequently be applied.

A key piece of HTS equipment is a plate: a small container, usually made of plastic, that features a grid of small, open divots called wells. Most of the wells contain experimentally useful matter, often a solution of dimethyl sulfoxide (DMSO) and some other chemical compound, the latter of which is different for each well across the plate. (The other wells are empty, intended for use as optional experimental controls.)

To prepare for an assay, the researcher fills each well of the plate with some biological entity that he or she wishes to conduct the experiment upon, such as a protein, some cells, or an animal embryo. After some incubation time has passed to allow the biological matter to absorb, bind to, or otherwise react (or fail to react) with the compounds in the wells, measurements are taken across all the plate's wells, either manually or by a machine. Manual measurements are often necessary when the researcher is using microscopy to (for example) seek changes or defects in embryonic development caused by the wells' compounds, looking for effects that a computer could not easily determine by itself. Otherwise, a specialized automated analysis machine can run a number of experiments on the wells (such as shining polarized light on them and measuring reflectivity, which can be an indication of protein binding). In this case, the machine outputs the result of each experiment as a grid of numeric values, with each number mapping to the value obtained from a single well. A high-capacity analysis machine can measure dozens of plates in the space of a few minutes like this, generating thousands of experimental datapoints very quickly.

Depending upon the results of this first assay, the researcher can perform follow up assays within the same screen by “cherrypicking” liquid from the wells that gave interesting results (known as “hits”) into new assay plates, and then re-running the experiment to collect further data on this narrowed set, confirming and refining observations.

A screening facility typically holds a library of stock plates, whose contents are carefully catalogued, and each of which may have been created by the lab or obtained from a commercial source. These stock plates themselves are not directly used in experiments; instead, separate assay plates are created as needed. An assay plate is simply a copy of a stock plate, created by pipetteing a small amount of liquid (often measured in nanoliters) from the wells of a stock plate to the corresponding wells of a completely empty plate.

Automation is an important element in HTS's usefulness. A specialized robot is often responsible for much of the process over the lifetime of a single assay plate, from creation through final analysis. An HTS robot can usually prepare and analyze many plates simultaneously, further speeding the data-collection process. HTS robots currently exist which can test up to 100,000 compounds per day (Hann 2004). Because many of the embodiments disclosed herein can be implemented for any such high throughput screening assays, such that the irradiation apparatus of the present invention constitues one or more units of a high throughput platform, it is possible to facilitate the overall process and reduce cost.

In some embodiments, the invention finds applications for a tissue microarray section. In the tissue microarray technique, a hollow needle is used to remove tissue cores as small as 0.6 mm in diameter from regions of interest in paraffin embedded tissues such as clinical biopsies or tumor samples. These tissue cores are then inserted in a recipient paraffin block in a precisely spaced, array pattern. Sections from this block are cut using a microtome, mounted on a microscope slide and then analyzed by any method of standard histological analysis. Each microarray block can be cut into 100-500 sections, which can be subjected to independent tests. Tests commonly employed in tissue microarray include immunohistochemistry, and fluorescent in situ hybridization. Tissue microarrays are particularly useful in analysis of cancer samples.

Typically, tissue microarrays (also TMAs) consist of paraffin blocks in which up to 1000 separate tissue cores are assembled in array fashion to allow simultaneous histological analysis. The major limitations in molecular clinical analysis of tissues using traditional histological methodology include the cumbersome nature of procedures, limited availability of diagnostic reagents and limited patient sample size. Subsequently, the technique of tissue microarray was developed to address these issues. Combining the features of the present invention with TMA, therefore, the technique can be further improved.

Similar approach can be taken to screen a collection of cells, proteins or genes. Thus, the invention also finds applications in the areas of MicroArray and Gene Expression (MAGE) and Cytomics, among others.

As used herein, the term “substrate” shall refer to any compartment or surface of support within which or onto which a sample or reagent may be placed. Many different types and many variations of substrates are contemplated, including but are not limited to: a capillary tube, a pipette tip, a needle, a cavity, a well, a chamber, a slide or a container, which are in some cases disposable. In some circumstances, especially when a sample volume is small, such as in a microlitter range, it may be desirable that the surface of a substrate that comes to a direct contact with a sample be coated. Surface coating on the substrate that coats or covers the paired conducting transmission lines to prevent nonspecific binding or adherence of a reagent, such as antibodies, dispersed in solution to the apparatus itself. Thin film/layer of coating on the order of micrometers such as Mylar film, Teflon, epoxy may be used to successfully prevent protein binding to the glass or gold aspects of the near-field radio frequency delivery applicator (e.g., antenna). Other materials may also be used, such as nonconductive silicone rubber, or silicone grease, for the same purpose.

In certain contexts, substrates may also refer to solid supports, onto which a molecule or molecules of interest may be coupled. In these cases, substrates may include beads, columns, filters, and the like.

The invention further contemplates embodimetns involving a staining or binding process achieved in a fluid stream of flow cytometry.

Flow cytometry is well known in the art and is a technique for counting, examining and sorting microscopic particles suspended in a stream of fluid. It allows simultaneous multiparametric analysis of the physical and/or chemical characteristics of single cells flowing through an optical and/or electronic detection apparatus. The present invention may be integrated into the technology to significantly improve results, both interms of time and quality.

In principle, flow cytometry uses a beam of light (usually laser light) of a single wavelength directed onto a hydro-dynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals found in the particle or attached to the particle may be excited into emitting light at a lower frequency than the light source. This combination of scattered and fluorescent light is picked up by the detectors, and by analysing fluctuations in brightness at each detector (one for each fluorescent emission peak) it is then possible to extrapolate various types of information about the physical and chemical structure of each individual particle. FSC correlates with the cell volume and SSC depends on the inner complexity of the particle (i.e. shape of the nucleus, the amount and type of cytoplasmic granules or the membrane roughness). Some flow cytometers on the market have eliminated the need for fluorescence and use only light scatter for measurement.

Modem flow cytometers are able to analyse several thousand particles every second, in “real time”, and can actively separate and isolate particles having specified properties. A flow cytometer is similar to a microscope, except that instead of producing an image of the cell, flow cytometry offers “high-throughput” (for a large number of cells) automated quantification of set parameters. To analyze solid tissues single-cell suspension must first be prepared.

A conventional flow cytometer has typically five main components: (1) a flow cell: liquid stream (sheath fluid) carries and aligns the cells so that they pass single file through the light beam for sensing; (2) a light source: commonly used are lamps (mercury, xenon); high power water-cooled lasers (argon, krypton, dye laser); low power air-cooled lasers (argon (488 nm), red-HeNe (633 nm), green-HeNe, HeCd (UV)); diode lasers (blue, green, red, violet); (3) a detector and Analogue to Digital Conversion (ADC) system: generating FSC and SSC as well as fluorescence signals; (4) an amplification system: linear or logarithmic; and (5) a computer for analysis of the signals. Modern instruments usually have multiple lasers and fluorescence detectors (the current record for a commercial instrument is 4 lasers and 18 fluorescence detectors). Increasing the number of lasers and detectors allows for multiple antibody labelling, and can more precisely identify a target population by their phenotype. The irradiation apparatus disclosed herein may be, for instance, incorporated prior to the step (1) shown above. Flow cytometers can also be configured as sorting instruments (fluorescent-activated cell sorting or FACS). As cells or particles pass through the instrument they can be selectively charged, based on user defined parameters, and can be deflected into separate paths of flow directed to different collection tubes. It is therefore possible to separate up to 4 defined populations of cells from an original mix with a high degree of accuracy and speed, which, in a conventional instrument is up to ˜90,000 cells per second in theory.

The present invention can further improve the capacities of these existing instruments by accelerating and enhancing one or more steps involving molecular intaractions discussed herein. Examples of instrument manufacturers include, but are not limted to the following: Amnis: ImageStream imaging flow cytometer (PC Platform); Bay bioscience corp: JSAN (PC platform); BD Biosciences: (FACS): FACSCalibur, FACScan, FACSort, FACSVantage (Mac OS platform) FACSCanto II, BD LSR II, FACSArray, FACSAria, FACSDiVa (PC Platform); Beckman Coulter (ex-Coulter): Cytomics FC500/FC500-MPL, Cell Lab Quanta SC, Cell Lab Vi-Cell, Epics XL/XL-MCL; Epics Altra (Hypersort) (PC platform); CytoBuoy: an instrument specialized for oceanographic applications; Cytopeia: Influx (PC platform); Dako (ex-Dako Cytomation): MoFlo, Cyan (IBM-PC platform); Fluid Imaging Technologies: FlowCAM® imaging flow cytometer and VisualSpreadsheet analysis software (PC platform); Guava Technologies: Personal Cell Analysis (PCA) System, Easycyte, Easycyte mini, PCA-96 (PC platform); Partec (for a period associated with Dako: PAS; CyFlow; CCA; PA (PC platform); PointCare Technologies: AuRICA; Accuri Cytometers: C6 Flow Cytometer™ System.

Accordingly, the cytometric technology has applications in a number of fields, including molecular biology, pathology, immunology, plant biology, marine biology and oceanography. As would be clear to a skilled artisan, in the field of molecular biology it is especially useful when used with fluorescence tagged antibodies, for instance, which provide information on specific characteristics of the cells being studied in the cytometer. It has broad application in medicine (especially in transplantation, heamatology, tumor immunology and chemotherapy, genetics). In marine biology, the auto-fluorescent properties of photosynthetic plankton can be exploited by flow cytometry in order to characterise abundance and community structure. In protein engineering, flow cytometry is used in conjunction with yeast display and bacterial display to identify cell surface-displayed protein variants with desired properties. Such technology may be used for measuring a wide range of parameters, including but are not limited to: volume and morphological complexity of cells; cell pigments such as chlorophyll or phycoerythrin; DNA (cell cycle analysis, cell kinetics, proliferation etc.); RNA; chromosome analysis and sorting (library construction, chromosome paint); protein expression and localization; transgenic products in vivo, particularly the Green fluorescent protein or related fluorescent proteins; cell surface antigens (Cluster of differentiation (CD) markers); intracellular antigens (various cytokines, secondary mediators etc.); nuclear antigens; enzymatic activity pH, intracellular ionized calcium, magnesium, membrane potential; membrane fluidity; apoptosis (quantification, measurement of DNA degradation, mitochondrial membrane potential, permeability changes, caspase activity); cell viability; monitoring electropermeabilization of cells; oxidative burst; characterising multidrug resistance (MDR) in cancer cells; glutathione; various combinations (DNA/surface antigens etc.).

Furthermore, using the technology disclosed herein, it is possible to facilitate the process of detecting, identifying and/or measuring a biological marker or markers, which are indicative of certain biological state. In some cases, the facilitated process of marker detection, identification and/or measurement will aid a prognostic or diagnostic process. Using the technology and the mehtods disclosed herein, a disease-assocciated antigen can be detected and measured from a biological sample, such as a tumor biopsy sample and a blood sample, in a fraction of time required for a conventional method. For example, the use of fluorescent label allows spatial determination of antigens or gene loci by examining localizations/distruibtions in cells or tissues, as well as compositional (phenotypic) information by detection, identification, and measurement by fluometric techniques, depending on specific purposes, which, one of ordinary skills in the art will be able to discern.

Additionally, the invention extends to facilitating certain therapeutic processes. For example, the process may involve controlled cross-linking of components of connective tissue (such as the lung) and smooth muscle constituents (arterial wall, aorta, etc.), including collagen and elastin. One exemplary application relates to keratoconus treatment. At present, cross linking by means of photosensitizers (Riboflavin) and UV light is used for the treatment of keratoconus. Keratoconus is a disease of the cornea that makes the cornea become weak and may gradually bulge outward. Approximately half of the keratoconus patients have significant visual problems beyond corrective lenses. The only resolution to keratoconus has been corneal transplantation, with a long healing period and unpredictable refractive error. Therefore, Corneal Cross Linking is used to increase the biomechanical stability of cornea to avoid corneal transplantation. This treatment generally involves Corneal Collagen Crosslinking with Riboflavin (C3-R), a one-time application of riboflavin eye drops to the eye. The riboflavin, when activated by approximately 30 minutes illumination with UV-A light, augments the collagen cross-links within the stroma and so recovers some of the cornea's mechanical strength. C3-R, developed at the Technische Universität Dresden, has been shown to slow or arrest the progression of keratoconus, and in some cases even reverse it, particularly when applied in combination with intracorneal ring segments. The methods presented herein thus may provide greater precision and fine control to improve such technique for keratoplasty.

The technology of the present invention may be incorporated into a number of clinical, medical, biochemical or analytical instruments. Non-limiting examples of such instruments include: an instrument for analyzing and/or measuring one or more parameters of a blood sample, a cardiac instrument or a kidney dialysis instrument.

The uses described herein are applicable to detect and identify an array of molecules of interest present in a variety of samples. Sample sources may include tissues, including, but not limited to lymph tissues; bodily fluids (e.g., blood, lymph fluid, etc.), cultured cells; cell lines; histological slides; tissue embedded in paraffin; etc. The term “tissue” as used herein refers to both localized and disseminated cell populations including, but not limited to: brain, heart, serum, breast, colon, bladder, epidernis, skin, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include, but are not limited to, blood, lymph fluid, cerebrospinal fluid, tears, saliva, urine, and feces, etc. In some embodiments, a sample comprises a blood or lymph node sample. Invasive and non-invasive techniques can be used to obtain such samples and are well documented in the art. A control cell sample may include a cell, a tissue, or may be a lysate of either. In some embodiments, a control sample may be a sample from a cell or subject that is free of cancer and/or free of a precancerous condition.

Apart from cell and tissue samples obtained from a subject, such as a human patient, the invention further contemplates its applications in the detection and identification of airborne pathogens using nucleic acid hybridization techniques or DNA mapping technology. In the latter, the technology includes a single molecule detection technology, where the irradiation apparatus and the methods for use disclosed herein may promote “open” or “elongated” conformation of nucleic acid molecules thereby enhancing the pathogen identification process. Such uses offer broad applications in rapid detection of airborne pathogens in an environmental sample. Typically, environmental samples are collected by filtering, and any airborne particulate matters may be dispersed into a suitable buffer and/or organic solvents.

In some embodiments, the non-covalent interaction comprises a polymerization process. As used herein, the term “polymerization process” refers to the process of forming or extending a matrix or matrices (e.g., gels that form nanoporous solids) comprising a matrix-forming molecule, optionally containing one or more components that catalyze or promote the formation process and/or enhance stability of a formed matrix. Examples of matrix-forming biomolecules include: certain polysaccharides such as agar, and certain proteins such as gelatin and collagen.

In some cases, the matrix-forming compounds comprise organic and or inorganic polymers. For example, the process of forming organic-inorganic polymer hybrids from various organic polymers such as poly(ethylene oxide) and poly(N-vinylpyrrolidone) can be accelerated with the assistance of microwave heating (such as 500 W, 2.45 GHz of microwave irradiation). Conventionally, such application of microwave irradiation was carried out with a standard household microwave oven, which in some cases produces uneven results, stemming from, presumably, uneven distribution of irradiation and lack of precise control. Therefore, the present invention may solve these technical limitations and provide faster, and better results.

In some cases, the polymerization process described above may envolve one or more extracellulat matrix (ECM) components. The ECM's main components are various glycoproteins, proteoglycans and hyaluronic acid. In most animals, the most abundant glycoproteins in the ECM are collagens. The ECM also contains many other components, including, proteins such as fibrin, elastin, fibronectins, laminins, and nidogens. Biological use relating to ECM components, polymerization thereof, in particular, would be apparent to those skilled in the art. As illustrated in FIG. 7 (Act 100), cell culture and tissue culture techniques often involve preparations and use of ECM as preferred substrates on which or into which cells and or tissues are grown and maintained. These also present valuable opportunities for tissue engineering. In this regard, the invention contemplates uses of microwave irradiation for improved polymerization of substrates, which may include, gelatin, elastin, collagen, fibrin, heparin and/or laminin. Such uses may relate to improved applications in the areas of skin grafting, wound healing, etc.

It is further contemplated that any of the methods or uses described herein may constitute one or more functional units in a high throughput screening process. High throughput screening is used to detect or identify spatial or compositional components of blood, cells or tissues, drug activity or cellular response to drug activity, cell identification, cell sorting, and tissue specific distribution of reagents applied therein.

Other applications of the present invention also include: use of microwave irradiation for enhancing DNA-small molecule interaction; use of microwave irradiation during chemical synthesis of DNA/oligonucleotides to achieve higher yields (by facilitated coupling of Phosphoramidite on Controlled Pore Glass); use of microwave irradiation for enhanced protein folding, such as denaturation and renaturation; use of microwave irradiation for improved detection of chromosomal abnormality, such as abnormal numbers of chromosomes and chromosomal translocations; preparation of improved matrices for tissue grafting or tissue engineering.

Apart from biomedical applications, the irradiators disclosed herein can find application in any situation in which a small volume of fluid or a thin tissue is to be irradiated by electromagnetic energy. A list of possible examples includes, but is not limited to, prototyping in large scale manufacturing processes in which RF energy is used, the food industry, electronics, aerospace, and other medical applications. Irradiator apparatus may be especially useful in chemical processes in which small, limited reagents are to be used.

Having thus described illustrative embodiments, it is to be appreciated that various alterations, modifications, and improvements will readily occur to those skilled in the art. Such alterations, modifications, and improvements are intended to be part of this disclosure, and are intended to be within the spirit and scope of this disclosure. While some examples presented herein involve specific combinations of functions or structural elements, it should be understood that those functions and elements may be combined in other ways according to the present invention to accomplish the same or different objectives. In particular, acts, elements, and features discussed in connection with one embodiment are not intended to be excluded from similar or other roles in other embodiments. Accordingly, the foregoing description and attached drawings are by way of example only, and are not intended to be limiting.

1. An apparatus, comprising:
a substrate; and
at least one electromagnetic field generator disposed on the substrate,
wherein the at least one electromagnetic field generator, when energized, is configured to deliver power only to a localized area comprising a thin region proximate to the substrate.

2. (canceled)

3. The apparatus of claim 1, wherein the thin region has a dimension of up to approximately 100 micrometers from and normal to a surface of the substrate.

4. The apparatus of claim 1, wherein the thin region has a dimension of at least approximately 100 micrometers from and normal to a surface of the substrate.

5. The apparatus of claim 1, wherein the at least one electromagnetic field generator is configured to deliver power to the thin region in an area parallel to a surface of the substrate having dimensions of approximately 8 millimeters by 8 millimeters.

6. (canceled)

7. (canceled)

8. The apparatus of claim 1, wherein the substrate includes at least one of a glass slide, a cavity, a well, a chamber, a capillary tube, a pipette tip, and a needle.

9. (canceled)

10. (canceled)

11. (canceled)

12. The apparatus of claim 1, wherein the at least one electromagnetic field generator comprises an array of conductors disposed on the substrate.

13. The apparatus of claim 12, wherein the array of conductors comprises a periodic array of equally spaced electrodes, and wherein an extent of the thin region from and normal to a surface of the substrate is based at least in part on a spacing between adjacent electrodes of the periodic array.

14. (canceled)

15. The apparatus of claim 13, further comprising at least one signal generator coupled to the equally spaced electrodes, such that when the at least one signal generator is energized, adjacent electrodes of the periodic array have opposite polarities.

16. The apparatus of claim 15, wherein adjacent electrodes of the periodic array have equal and opposite potentials.

17. The apparatus of claim 15, wherein the at least one signal generator, when energized, is configured to provide up to approximately 20 dBm of power.

18. The apparatus of claim 15, wherein the at least one signal generator is disposed on the substrate proximate to the at least one electromagnetic field generator.

19. The apparatus of claim 18, wherein the at least one signal generator is coupled to the at least one electromagnetic field generator via a flip-chip pressure connector.

20. (canceled)

21. The apparatus of claim 12, wherein the array of electrodes comprises a coiled transmission line.

22. The apparatus of claim 21, wherein the coiled transmission line is configured to have an octagonal shape.

23. The apparatus of claim 21, wherein each electrode in the array has a line width of approximately 100 micrometers, and wherein adjacent electrodes of the array are separated by a distance of approximately 100 micrometers.

24. The apparatus of claim 1, wherein the power is delivered to the thin region via a magnetic field above the electrodes, and wherein the at least one electromagnetic field generator comprises a length of wire arranged in a serpentine pattern.

25. (canceled)

26. A medical or diagnostic instrument, comprising:
the apparatus of any of the foregoing claims; and
at least one sensor configured to measure at least one characteristic of at least one sample located in the thin region and subjected to the power delivered to the thin region.

27. An electromagnetic irradiation method, comprising an act of:
A) delivering power only to a localized area comprising a thin region proximate to a substrate.

28. The method of claim 27, wherein the power is in a range of up to approximately 100 milliwatts.

29. The method of claim 27, wherein the thin region has a dimension of up to approximately 100 micrometers from and normal to a surface of the substrate.

30. The method of claim 27, wherein the thin region includes an area above the substrate having dimensions of approximately 8 millimeters by 8 millimeters.

31. The method of claim 27, wherein the act A) comprises an act of:
delivering the power to the thin region over a frequency range from DC to tens of gigahertz.

32. The method of claim 27, wherein the act A) comprises and act of:
delivering the power to the thin region via microwave radiation.

33. The method of claim 27, wherein the thin region above the substrate includes at least one sample, and wherein the act A) comprises an act of:
B) delivering the power to the at least one sample.

34. The method of claim 33, wherein the at least one sample includes at least one organic molecule.

35. The method of claim 33, wherein the at least one sample includes a volume of fluid.

36. The method of claim 33, wherein the at least one sample includes at least one of a pH buffer solution, an osmotic balance solution, and a salt solution.

37. The method of claim 33, wherein the at least one sample includes at least one biological sample.

38. The method of claim 33, wherein the at least one sample includes at least one tissue sample.

39. The method of claim 38, wherein the at least one tissue sample includes at least one stained tissue sample.

40. The method of claim 33, wherein the at least one sample includes at least one magnetic particle.

41. The method of claim 33, wherein the act B) comprises an act of:
delivering the power to the at least one sample so as to facilitate at least one chemical process in the at least one sample.

42. The method of claim 33, wherein the act B) comprises an act of:
exciting an electric mode in the at least one sample.

43. The method of claim 33, wherein the act B) comprises an act of:
exciting a magnetic mode in the at least one sample.

44. The method of claim 33, wherein the act B) comprises an act of:
independently exciting a magnetic mode and an electric mode in the at least one sample.

45-107. (canceled)
